(19) United States
(12) Patent Application Publication
(10) Pub. No.: US 2023/0016068 A1
(43) Pub. Date: Jan. 19, 2023

(54) BENDING SECTION OF ENDOSCOPE, ENDOSCOPE INSERTION SECTION, AND ENDOSCOPE

(71) Applicant: OLYMPUS CORPORATION, Tokyo (JP)

(72) Inventors: Eijiro SATO, Tokyo (JP); Eiji MATSUDA, Sagamihara-shi (JP); Ryo HASEGAWA, Tokyo (JP); Takashi NARA, Tokyo (JP)

(73) Assignee: OLYMPUS CORPORATION, Tokyo (JP)

(21) Appl. No.: 17/954,797

(22) Filed: Sep. 28, 2022

Related U.S. Application Data
(63) Continuation of application No. PCT/JP2020/014614, filed on Mar. 30, 2020.

(51) Int. Cl. A61B 1/005

(52) U.S. Cl. CPC A61B 1/0055; A61B 1/0057

(57) ABSTRACT

A bending section of an endoscope includes a first bending piece and a second bending piece in each of which a hole is formed, the first bending piece includes a first groove communicating with the hole and formed toward an outer side of a radial direction, the second bending piece is disposed adjacent to the first bending piece and includes a second groove formed from an outer circumference of the second bending piece toward the hole, and the wire is disposed in the first groove and the second groove.

### CROSS REFERENCE TO RELATED APPLICATION

This application is a continuation application of PCT/JP2020/014614 filed on Mar. 30, 2020, the entire contents of which are incorporated herein by this reference.

### BACKGROUND OF THE INVENTION

### 1. Field of the Invention

The present invention relates to a bending section of an endoscope provided in an endoscope insertion section, the bending section being bent by a wire being towed, an endoscope insertion section, and an endoscope.

### 2. Description of the Related Art

In recent years, endoscopes have been widely used in a medical field and an industrial field. With endoscopes, observation, treatment, and the like of a site to be examined in a subject can be performed by inserting an elongated insertion section into the subject.

A configuration is well known in which a bending section bendable in a plurality of directions is provided on a distal end side in an insertion section of an endoscope.

Besides improving an advancing property of the insertion section in the bending section in a conduit, the bending section changes an observing direction of an observation optical system provided at a distal end portion located further forward than the bending section in the insertion section.

The bending section includes, for example, a plurality of bending pieces respectively having predetermined lengths along a longitudinal axis of the insertion section and including tubular thick portions.

As the bending section, a configuration is well known in which a plurality of bending pieces adjacent to each other in a direction along a longitudinal axis (hereinafter referred to as longitudinal axial direction) of the bending pieces are coupled via a plurality of turnable rivets for bending the bending section in an up-down direction and the plurality of turnable rivets for bending the bending section in a left-right direction to thereby be bendable in upward, downward, left, and right four directions.

Note that two pairs of, that is, four towing wires (hereinafter simply referred to as wires) movable to a front and a rear in the longitudinal axial direction, distal ends of the towing wires being fixed to the bending piece located closest to a. distal end side among the plurality of bending pieces, are inserted through the insertion section.

Any one of the four wires is towed from the operation section of the endoscope, whereby the bending section is bendable in any one of the upward, downward, left, and right directions.

In the bending section, a configuration is also well known in which the bending pieces adjacent to each other in the longitudinal axial direction are coupled by a pair of rivets and are bendable in any one of the upward and downward two directions or the left and right two directions by a pair of, that is, two wires inserted through the insertion section.

Here, in a configuration in which an insertion section having a small diameter equal to or smaller than 5 mm or 3 mm such as a pyeloureteroscope is requested in the endoscope used in the medical field, it is well known that, in the configuration in which the rivets are used explained above, it is difficult to reduce a diameter of the bending section because the rivets are used.

Therefore, for example, Japanese Patent Application Laid-Open Publication No. 2005-7068 discloses a configuration of a bending section including a rivet-less structure in which, in order to realize a reduction in a diameter of the bending section, a plurality of bending pieces, contact sections provided at end portions of which directed in a longitudinal axial direction are in contact with one another, the plurality of bending pieces being capable of turning with respect to one another, are consecutively connected in the longitudinal axial direction and four wires pierce through annular thick portions of the respective bending pieces, whereby the annular thick portions function as wire receivers.

Note that, in the rivet-less structure as well, a configuration is well known in which two wires pierce through the annular thick portions of the respective bending pieces in order to bend the bending section in two directions.

### SUMMARY OF THE INVENTION

A bending section of an endoscope according to an aspect of the present invention is a bending section of an endoscope bent by a wire being towed, the bending section being provided in an endoscope insertion section, the bending section including at least one first bending piece and at least one second bending piece, each of the first and second bending pieces being tubular and including a hole formed along a longitudinal axis of the endoscope insertion section, an internal component of the endoscope being disposed in the hole, wherein the first bending piece includes a first groove communicating with the hole, formed from an inner circumference of the first bending piece toward an outside of a radial direction of the longitudinal axis, and formed at a width substantially equal to an outer diameter of the wire and a depth equal to or larger than the outer diameter of the wire, the second bending piece is disposed adjacent to the first bending piece along the longitudinal axis, and the second bending piece includes a second groove formed from an outer circumference of the second bending piece toward the hole and formed at a width substantially equal to the outer diameter of the wire and a depth equal to or larger than the outer diameter of the wire, and the wire is disposed in the first groove and the second groove.

An endoscope insertion section according to an aspect of the present invention is an endoscope insertion section funned in a tube shape, the endoscope insertion section including a bending section bent by a wire being towed and including at least one first bending piece and at least one second bending piece, each of the first and second bending pieces being tubular and including a hole formed along a longitudinal axis of the endoscope insertion section, an internal component of the endoscope being disposed in the hole, wherein the first bending piece includes a first groove communicating with the hole, formed from an inner circumference of the first bending piece toward an outer side of a radial direction of the longitudinal axis, and formed at a width substantially equal to an outer diameter of the wire and a depth equal to or larger than the outer diameter of the wire, the second bending piece is disposed adjacent to the first bending piece along the longitudinal axis, and the second bending piece includes a second groove formed from an outer circumference of the second bending piece toward the hole and formed at a width substantially equal to the outer diameter of the wire and a depth equal to or larger than the outer diameter of the wire, and the wire is disposed in the first groove and the second groove.

Further, an endoscope according to an aspect of the present invention is an endoscope including an endoscope insertion section formed in a tube shape, the endoscope insertion section including a bending section bent by a wire being towed and including at least one first bending piece and at least one second bending piece, each of the first and second bending pieces being tubular and including a hole formed along a longitudinal axis of the endoscope insertion section, an internal component of the endoscope being disposed in the hole, wherein the first bending piece includes a first groove communicating with the hole, formed from an inner circumference of the first bending piece toward an outer side of a radial direction of the longitudinal axis, and formed at a width substantially equal to an outer diameter of the wire and a depth equal to or larger than the outer diameter of the wire, the second bending piece is disposed adjacent to the first bending piece along the longitudinal axis, and the second bending piece includes a second groove formed from an outer circumference of the second bending piece toward the hole and formed at a width substantially equal to the outer diameter of the wire and a depth equal to or larger than the outer diameter of the wire, and the wire is disposed in the first groove and the second groove.

### BRIEF DESCRIPTION OF THE DRAWINGS

FIG. 1 is a partial perspective view showing an endoscope including, in an endoscope insertion section, a bending section of the endoscope in a first embodiment;

FIG. 2 is a partial cross-sectional view showing, together with two wires, a plurality of bending pieces configuring a first bending section;

FIG. 3 is a diagram showing, together with the wires, a cross section of a first bending piece of the bending section taken along a III-III line in FIG. 2;

FIG. 4 is a diagram showing, together with the wires, a cross section of a second bending piece of the bending section taken along a IV-IV line in FIG. 2;

FIG. 5 is a cross-sectional view showing a state in which the first bending piece rotates in an upward direction up to a maximum rotation angle with respect to the second bending piece shown in FIG. 2;

FIG. 6 is a cross-sectional view showing a state in which a first bending piece rotates in an upward direction up to a maximum rotation angle with respect to a second bending piece in a bending piece configuring a bending section of an endoscope in a second embodiment;

FIG. 7 is a cross-sectional view showing a modification of a shape of a second groove of the second bending piece shown in FIG. 6;

FIG. 8 is a cross-sectional view showing a modification of a shape of a first groove of the first bending piece shown in FIG. 6;

FIG. 9 is a cross-sectional view showing a modification in which respective distal end portions and respective proximal end portions in the first groove of the first bending piece and the second groove of the second bending piece shown in FIG. 6 are formed as cutouts;

FIG. 10 is a cross-sectional view showing a modification in which respective distal end portions and respective proximal end portions in the first groove of the first bending piece and the second groove of the second bending piece shown in FIG. 7 are formed as cutouts;

FIG. 11 is a cross-sectional view showing a modification in which respective distal end portions and respective proximal end portions in the first groove of the first bending piece and the second groove of the second bending piece shown in FIG. 8 are formed as cutouts

FIG. 12 is a partial cross-sectional view showing, together with two wires, a plurality of bending pieces configuring a bending section in a third embodiment;

FIG. 13 is a diagram showing, together with the wires, side by side with a cross section of a second bending piece taken along a XIII′-XIII′ line in FIG. 12, a cross section of a first bending piece taken along a XIII-XIII line in FIG. 12;

FIG. 14 is a partial cross-sectional view of a bending section of a general endoscope;

FIG. 15 is a partial cross-sectional view showing a plurality of bending pieces configuring a bending section of the configuration;

FIG. 16 is a perspective view of one bending piece shown in FIG. 15;

FIG. 17 is a perspective view of the other bending piece shown in FIG. 15;

FIG. 18 is a partial cross-sectional view in a coupling part of an active bending section and a passive bending section in the bending section of the configuration;

FIG. 19 is a main part enlarged perspective view enlarging and showing a schematic configuration of an endoscope distal end portion in a fourth embodiment;

FIG. 20 is a side view of the endoscope distal end portion shown in FIG. 19;

FIG. 21 is an exploded perspective view showing a state in which a distal end cover is removed at the endoscope distal end portion shown in FIG. 19;

FIG. 22 is a main part enlarged perspective view enlarging and showing a schematic configuration of an endoscope distal end portion in a fifth embodiment;

FIG. 23 is a side view of the endoscope distal end portion shown in FIG. 22;

FIG. 24 is an exploded perspective view showing a state in which a distal end cover is removed at the endoscope distal end portion shown in FIG. 22;

FIG. 25 is a main part enlarged exterior perspective view showing an exterior of an endoscope distal end portion in a sixth embodiment;

FIG. 26 is a side view showing a vicinity of a distal end portion of a light guide cable in a form applied to the endoscope distal end portion in the sixth embodiment;

FIG. 27 is a side view showing a vicinity of a distal end portion of a light guide cable in another form applied to the endoscope distal end portion in the sixth embodiment;

FIG. 28 is a cross-sectional view showing a cut surface indicated by an alternate long and two short dashes line in FIG. 25 viewed in an arrow [28] direction;

FIG. 29 is a schematic perspective view showing a part of an internal structure of an endoscope operation section;

FIG. 30 is a cross-sectional view showing a configuration example in which a sleeve at a distal end of a wire is connected to a distal end bending piece in a seventh embodiment;

FIG. 31 is a perspective view showing a configuration example of a cutout hole provided in the distal end bending piece for inserting through the wire in the seventh embodiment;

FIG. 32 is a cross-sectional view showing a configuration example in which a sleeve provided at a distal end of the wire is heat-welded to a bending section outer skin through the cutout hole in a first modification of the seventh embodiment;

FIG. 33 is a cross-sectional view showing a configuration example in which the sleeve provided at the distal end of the wire is pressed into an attachment hole provided in the distal end bending piece in a second modification of the seventh embodiment;

FIG. 34 is a cross-sectional view showing a configuration example in which the sleeve provided at the distal end of the wire is restricted from moving to a distal end side in an insertion axial direction by a restricting member in a third modification of the seventh embodiment;

FIG. 35 is a cross-sectional view showing a configuration example of a rough surface provided on a surface of a distal end bending piece made of metal in order to heat-weld a bending section outer skin in an eighth embodiment;

FIG. 36 is a cross-sectional view showing a configuration example in which a cut surface is provided on a rear end side of a circumferential convex portion of the distal end bending piece in the eighth embodiment;

FIG. 37 is a cross-sectional view showing a configuration example in which a bending section outer skin is sandwiched by a connection tube and the distal end bending piece in a modification of the eighth embodiment;

FIG. 38 is a cross-sectional view showing a configuration example of a passive bending section provided between a bending section and a flexible tube section in a ninth embodiment;

FIG. 39 is a perspective view showing a configuration example of a three-layer flexible tube in the ninth embodiment;

FIG. 40 is a cross-sectional view showing the configuration example of the three-layer flexible tube in the ninth embodiment;

FIG. 41 is a cross-sectional view showing an example in which the passive bending section is configured by covering the three-layer flexible tube with a passive bending section outer skin in the ninth embodiment;

FIG. 42 is a cross-sectional view showing a configuration example in which a distal end portion of the three-layer flexible tube is connected to an outer circumferential surface of a rear end bending piece and a rear end portion of the three-layer flexible tube is connected to an outer circumferential surface of a front pipe sleeve of the flexible tube section in the ninth embodiment;

FIG. 43 is a cross-sectional view showing a configuration example in which the connection of the distal end portion of the three-layer flexible tube and the outer circumferential surface of the rear end bending piece and the connection of the rear end portion of the three-layer flexible tube and the outer circumferential surface of the front pipe sleeve of the flexible tube section are performed by laser welding;

FIG. 44 is a cross-sectional view showing a configuration example in which the distal end portion of the three-layer flexible tube is connected to an inner circumferential surface of the rear end bending piece and the rear end portion of the three-layer flexible tube is connected to an inner circumferential surface of the front pipe sleeve of the flexible tube section;

FIG. 45 is a cross-sectional view showing a configuration example in which the rear end bending piece is insert-molded in a metal pipe and the three-layer flexible tube of the passive bending section is laser-welded to the metal pipe in a first modification of the ninth embodiment;

FIG. 46 is a cross-sectional view showing a configuration example in which a two-layer flexible tube and a heat shrinkable tube are used in the passive bending section in a second modification of the ninth embodiment;

FIG. 47 is a side view showing a configuration example of a bending section braid in a tenth embodiment;

FIG. 48 is a diagram showing a processing example for manufacturing the bending section braid in a modification of the tenth embodiment;

FIG. 49 is a cross-sectional view showing a configuration example in which a flexible tube section braid is internally inserted into a front pipe sleeve of a flexible tube section and the flexible tube section braid is locked in an outer diameter direction by a taper member in an eleventh embodiment;

FIG. 50 is a cross-sectional view showing a configuration example in which the flexible tube section braid is externally inserted onto the front pipe sleeve of the flexible tube section and the flexible tube section braid is locked in an inner diameter direction by a resin tube in a modification of the eleventh embodiment: and

FIG. 51 is a cross-sectional view showing a configuration example in which a connection tube and a bending section braid are laser-welded to a distal end bending piece in a twelfth embodiment.

### DETAILED DESCRIPTION OF THE PREFERRED EMBODIMENTS

In general, a conventional bending section of an endoscope in which wires are configured to pierce through annular thick portions of respective bending pieces has a configuration in which the thick portions have a predetermined thickness and project from inner circumferences of the bending pieces to a radial direction inner side of a longitudinal axis.

Accordingly, if it is attempted to achieve a further reduction in a diameter of the bending section, since the thick portions are present, there is a problem in that a housing space for internal components that can be built in the bending section decreases and performance of the internal components is limited, more specifically, a problem in that, for example, size of a known channel decreases. In view of such a problem, if it is attempted to secure, in the same size as the conventional one, the housing space for the internal components that can be built in the bending section, there is still a problem in that the bending section is increased in size by the thick portions.

Accordingly, a configuration in which the thick portions of the respective bending pieces are formed thin is conceivable. However, when the respective bending pieces are formed from resin, there is also a problem in that the thick portions cannot be formed thin because of a molding problem.

Note that the problems described above are the same when wire receivers are not formed integrally with the bending pieces and are separate.

Further, in all of a rivet structure, a rivet-less structure, and a structure in which a well-known bending section includes a tubular member of a super elastic alloy such as nitinol, wires are configured to be pierced through through-holes of a plurality of wire receivers of the respective bending pieces or the tubular member from a front or a rear one by one over an entire length in a longitudinal axial direction of the bending section. Therefore, there is also a problem in that it takes time to assemble the wires, that is, assembly of the wires to the bending section is poor and manufacturing cost increases.

According to the present invention, it is possible to provide a bending section of an endoscope, an endoscope insertion section, and an endoscope including a configuration in which it is possible to secure a housing space for internal components as large as possible while achieving a reduction in a diameter and assembly of wires is improved.

Embodiments of the present invention are explained below with reference to the drawings. Note that, in the embodiments explained below, an endoscope is explained using a pyeloureteroscope as an example.

### First Embodiment

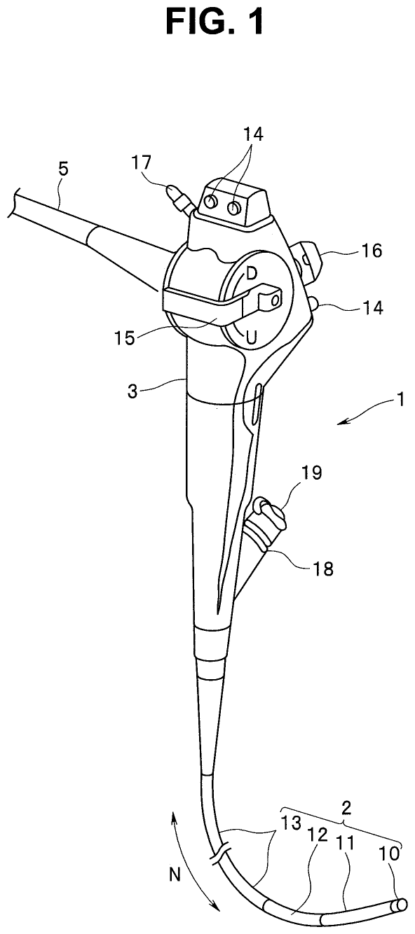

FIG. 1 is a partial perspective view showing an endoscope including, in an endoscope insertion section, a bending section of the endoscope in the present embodiment.

As shown in FIG. 1, a main part of an endoscope 1 is configured by a tubular endoscope insertion section (hereinafter simply referred to as insertion section) 2 elongated in a direction along a longitudinal axis (hereinafter referred to as longitudinal axial direction) N and having flexibility, an operation section 3 provided on a proximal end side of the insertion section 2, a universal cord 5 extending from the operation section 3, and a not-shown connector provided at an extension end of the universal cord 5 and connected to a not-shown image processing apparatus, a not-shown light source apparatus, and the like.

A main part of the insertion section 2 is configured by, in order from a distal end side, a rigid distal end portion 10 including a not-shown observation optical system and the like inside, a bending section 11, which is an active bending section consecutively connected to a proximal end side of the distal end portion 10 and actively bendable in a plurality of directions, for example, upward (U) and downward (D) two directions, a passive bending section 12 consecutively connected to a proximal end side of the bending section 11 and passively bendable in a plurality of directions, and a soft flexible tube section 13 consecutively connected to a proximal end side of the passive bending section 12 and having flexibility.

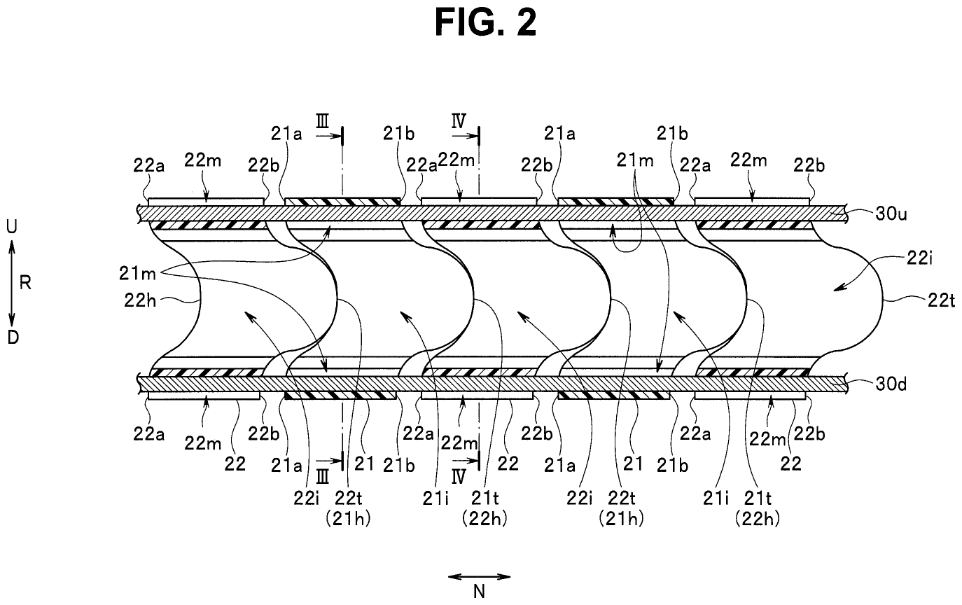

Note that the bending section 11 is bent in the upward or downward direction by one of wires 30u and 30d (for both of which, see FIG. 2) explained below being towed according to operation of a bending operation lever 15 explained below.

The bending section 11 may be configured to be bendable in the left and right two directions or may be configured to be bendable in the upward, downward, left, and right four directions, or the upward, downward, left, and right composite directions.

Further, in the present embodiment, a configuration in which the passive bending section 12 is provided in the endoscope 1 is explained as an example. However, the flexible tube section 13 may be directly consecutively connected to the proximal end side of the bending section 11.

Remote switches 14 for performing, for example, an image control instruction such as freeze or release, a bending operation lever 15 for bending operation of the bending section 11, a suction button 16 for performing suction operation, a suction pipe sleeve 17 communicating with a not-shown suction channel provided in the insertion section 2, and the like are provided on a proximal end side of the operation section 3.

Further, a treatment instrument insertion opening 18 for inserting a treatment instrument such as forceps into the suction channel is provided on a distal end side of the operation section 3. A forceps plug 19 is detachably attachable to the treatment instrument insertion opening 18.

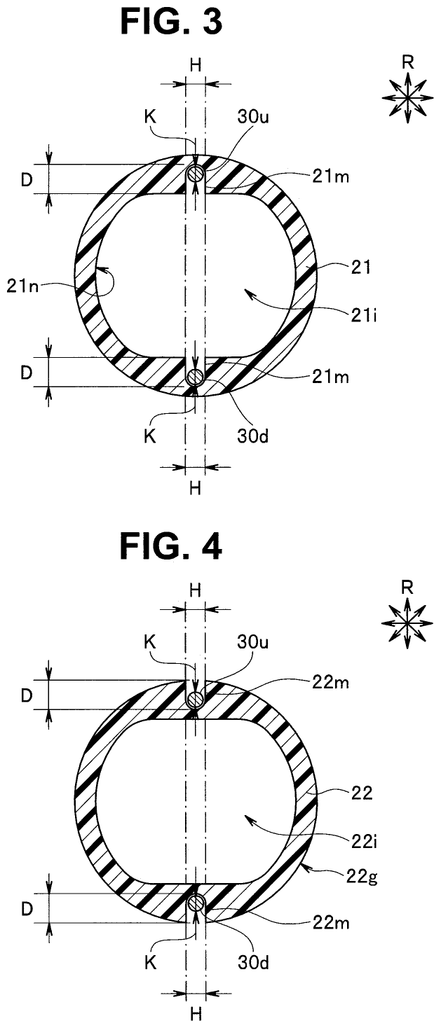

Subsequently, a configuration of the bending section 11 is explained with reference to FIG. 2 to FIG. 4. FIG. 2 is a partial cross-sectional view showing, together with two wires, a plurality of bending pieces configuring the bending section shown in FIG. 1. FIG. 3 is a diagram showing, together with the wires, a cross section of a first bending piece of the bending section taken along a III-III line in FIG. 2. FIG. 4 is a diagram showing, together with the wires, a cross section of a second bending piece of the bending section taken along a IV-IV line in FIG. 2.

As shown in FIG. 2, in the present embodiment, the bending section 11 is configured by a plurality of first bending pieces 21 and a plurality of second bending pieces 22 being coupled in the longitudinal axial direction N.

More specifically, the first bending pieces 21 and the second bending pieces 22 are disposed to be coupled adjacent to each other in the longitudinal axial direction N.

More specifically, the first bending pieces 21 and the second bending pieces 22 are disposed to be alternately coupled in the longitudinal axial direction N in such a manner as the first bending piece 21, the second bending piece 22, the first bending piece 21, the second bending piece 22 . . . in the longitudinal axial direction N.

Note that, in FIG. 2, as an example, two first bending pieces 21 and three second bending pieces 22 are alternately coupled in the longitudinal axial direction N. However, the numbers of the first bending pieces 21 and the second bending pieces 22 are not limited to these number.

In other words, the numbers of the first bending pieces 21 and the second bending pieces 22 may be one or may be plural. The numbers of the first bending pieces 21 and the second bending pieces 22 may be the same or may be different.

A known braid and a known bending rubber cover outer circumferences of the first bending piece 21 and the second bending piece 22. However, in FIG. 2, the braid and the bending rubber are omitted to simplify the drawing.

The first bending piece 21 is formed in a tubular shape by a hole 21i, that has a predetermined length in the longitudinal axial direction N and pierces through in the longitudinal axial direction and in which various known internal components of the bending section 11 such as a light guide cable and an image pickup cable are disposed inside, being formed. Note that the first bending piece 21 is formed from, for example, resin.

The second bending piece 22 is also formed in a tubular shape by a hole 22i, that has a predetermined length in the longitudinal axial direction N and that pierces through in the longitudinal axial direction and in which the various known internal components are disposed inside, being formed. Note that the second bending piece 22 is formed from, for example, resin.

Note that the first bending piece 21 and the second bending piece 22 are formed in the same outer diameter in a radial direction R of the longitudinal axis. The first bending piece 21 and the second bending piece 22 may be formed in the same length or may be formed in different lengths in the longitudinal axial direction N.

The first bending piece 21 includes, on a proximal end face, which is an end portion in the longitudinal axial direction N, a pair of semicircular convex portions 21t extending to a rear in the longitudinal axial direction N and symmetrical with respect to a center axis of the first bending piece 21.

Further, the first bending piece 21 includes, on a distal end face, which is an end portion in the longitudinal axial direction N, a pair of semicircular concave portions 21h recessed to the rear in the longitudinal axial direction N and symmetrical with respect to the center axis of the first bending piece 21.

The second bending piece 22 includes, on a proximal end face, which is an end portion in the longitudinal axial direction N, a pair of semicircular convex portions 22t extending to the rear in the longitudinal axial direction N and symmetrical with respect to a center axis of the second bending piece 22.

Further, the second bending piece 22 includes, on a distal end face, which is an end portion in the longitudinal axial direction N, a pair of semicircular concave portions 22h recessed to the rear in the longitudinal axial direction N and symmetrical with respect to the center axis of the second bending piece 22.

Since the first bending piece 21 and the second bending piece 22 are alternately disposed in the longitudinal axial direction N, the pair of convex portions 22t of the second bending piece 22 is in contact with the pair of concave portions 21h of the first bending piece 21 and the pair of convex portions 21t of the first bending piece 21 is in contact with the pair of concave portions 22h of the second bending piece 22, . . . .

Note that the contact of the first bending piece 21 and the second bending piece 22 is performed in a state in which the first bending piece 21 and the second bending piece 22 are compressed (a state in which the first bending piece 21 and the second bending piece 22 are cut in) in the longitudinal axial direction N by the wires 30u and 30d explained below.

Consequently, the first bending piece 21 and the second bending piece 22 are configured such that bending pieces adjacent to each other in the longitudinal axial direction N are capable of turning with respect to each other.

More specifically, the first bending piece 21 and the second bending piece 22 adjacent to each other in the longitudinal axial direction N are in contact to be capable of turning in the up-down direction (UD).

Accordingly, for example, when the wire 30u explained below is towed to the rear, the pair of convex portions 21t in contact with the pair of concave portions 22h rotates in the upward direction (U) and the pair of convex portions 22t in contact with the pair of concave portions 21h rotates in the upward direction, whereby the bending section 11 bends in the upward direction (U) in FIG. 2.

Note that a maximum rotation angle in the upward direction (U) of the pair of convex portions 21t in contact with the pair of concave portions 22h is specified by a shoulder portion 21b at a proximal end of the first bending piece 21 coming into contact with a shoulder portion 22a at a distal end of the second bending piece 22. A maximum rotation angle in the upward direction (U) of the pair of convex portions 22t in contact with the pair of concave portions 21h is specified by a shoulder portion 22b at a proximal end of the second bending piece 22 coming into contact with a. shoulder portion 21a at a distal end of the first bending piece 21.

On the contrary, when the wire 30d explained below is towed to the rear, the pair of convex portions 21t in contact with the pair of concave portions 22h rotates in the downward direction and the pair of convex portions 22t in contact with the pair of concave portions 21h rotates in the downward direction, whereby the bending section 11 bends in the downward direction (D) in FIG. 2.

Note that a maximum rotation angle in the downward direction (D) of the pair of convex portions 21t in contact with the pair of concave portions 22h is specified by the shoulder portion 21h at the proximal end of the first bending piece 21 coming into contact with the shoulder portion 22a at the distal end of the second bending piece 22. A maximum rotation angle in the downward direction (D) of the pair of convex portions 22t in contact with the pair of concave portions 21h is specified by the shoulder portion 22b at the proximal end of the second bending piece 22 coming into contact with the shoulder portion 21a at the distal end of the first bending piece 21.

In other words, the bending section 11 in the present embodiment has a known rivet-less structure in which a rivet is not used for coupling of the first bending piece 21 and the second bending piece 22.

Here, as shown in FIG. 3, in an inner circumference 21n of the first bending piece 21, two first grooves 21m communicating with the hole 21i and extending from the inner circumference 21n toward an outer side in the radial direction R are formed in positions opposed to each other, for example, in the up-down direction (UD).

The first grooves 21m have a width H substantially equal to an outer diameter K of the wires 30u and 30d explained below and are formed at a depth D equal to or larger than the outer diameter K and are further formed in the longitudinal axial direction N from the distal end to the proximal end of the first bending piece 21. Note that the first grooves 21m are formed at the constant depth D from the distal end to the proximal end of the first bending piece 21. According to the formation of the first grooves 21m, a wall surface is formed in the first bending piece 21 in a direction turned approximately 90° from the up-down direction (UD) from the distal end to the proximal end in the longitudinal axial direction N.

The wires 30u and 30d can be inserted into and pulled out from the first grooves 21m in the radial direction R from the hole 21i.

As shown in FIG. 4, in an outer circumference 22g of the second bending piece 22. two second grooves 22m are formed from the outer circumference 22g toward the hole 22i in positions opposed to each other, for example, in the up-down direction (UD).

The second grooves 22m have the width H substantially equal to the outer diameter K of the wires 30u and 30d explained below and are formed at the depth D equal to or larger than the outer diameter K and are further formed in the longitudinal axial direction N from the distal end to the proximal end of the second bending piece 22. Note that the second grooves 22m are formed at the constant depth D from the distal end to the proximal end of the second bending piece 22. According to the formation of the second grooves 22m, a wall surface is formed in the second bending piece 22 in a direction turned approximately 90° from the up-down direction (UD) from the distal end to the proximal end in the longitudinal axial direction N.

Note that, in the present embodiment, the width H and the depth D of the first grooves 21m are the same as the width H and the depth D of the second grooves 22m.

The wires 30u and 30d can be inserted into and pulled out from the second grooves 22m from the outer side in the radial direction R.

The two wires 30u and 30d that turn the first bending piece 21 and the second bending piece 22 by towing as explained above are disposed in the first grooves 21m and the second grooves 22m.

More specifically, the wire 30u is disposed in the longitudinal axial direction N in the first groove 21m and the second groove 22m located in the upward direction (U) and the wire 30d is disposed in the longitudinal axial direction N in the first groove 21m and the second groove 22m located in the downward direction (D).

Note that distal ends of the wires 30u and 30d are connected to, of the first bending pieces 21 and the second bending pieces 22 coupled in the longitudinal axial direction N, the bending pieces located closest to the distal end side. Proximal ends of the wires 30u and 30d are connected to a pulley or the like turned by the bending operation lever 15.

As work for inserting the wires 30u and 30d through the first grooves 21m and the second grooves 22m, first, the wires 30u and 30d are inserted through the holes 21i of the plurality of first bending pieces 21 from the front or the rear in the longitudinal axial direction N.

Thereafter, by applying tension to the wires 30u and 30d in the longitudinal axial direction N, the wires 30u and 30d are moved from the holes 21i in the radial direction R and respectively fit in the upper and lower first grooves 21m.

Finally, the second bending pieces 22 are disposed in gaps among the first bending pieces 21 in the longitudinal axial direction such that the wires 30u and 30d are fit in the second grooves 22m from the outer side in the radial direction R. In this way, the first bending pieces 21 and the second bending piece 22 are coupled in a rivet-less manner and disposed.

Note that other components and an assembly method of the bending section 11 are the same as components and an assembly method of a bending section used in a conventional rivet-less bending section structure,

As explained above, in the present embodiment, the wires 30u and 30d are explained as being able to be inserted into and pulled out from the first grooves 21m of the first bending piece 21 and the second grooves 22m of the second bending piece 22 in the radial direction R.

The bending section 11 is explained as being configured by coupling, in the longitudinal axial direction N, the first bending piece 21 in which the wires 30u and 30d can be inserted into and pulled out from the first grooves 21m on the inner side in the radial direction R and the second bending piece 22 in which the wires 30u and 30d can be inserted into and pulled out from the second grooves 22m from the outer side in the radial direction R.

According to the above, if the first bending piece 21 and the second bending piece 22 are alternately coupled in the longitudinal axial direction N or if the first bending piece 21 and the second bending piece 22 are coupled at a predetermined interval in the longitudinal axial direction N, the second grooves 22m can prevent the wires 30u and 30d from coming off to the inner side (the hole 21i side) and the first grooves 21m can prevent the wires 30u and 30d from corning off to the outer side in the radial direction R.

Since the first grooves 21m communicate with the hole 21i, it is unnecessary to provide, on the inner circumference 21n side, a conventional thick portion having sufficient thickness including a through-hole through which the wires 30u and 30d are inserted. Therefore, the first bending piece 21 can be reduced in diameter.

Further, since the second grooves 22m communicate with the outside, it is unnecessary to provide, on the outer circumference 22g side, the conventional thick portion having sufficient thickness including a through-hole through which the wires 30u and 30d. are inserted. Therefore, the second bending piece 22 can be reduced in diameter.

In other words, the bending section 11 can be reduced in diameter by the first bending piece 21 and the second bending piece 22 reduced in diameter. Even if the reduction in the diameter of the bending section 11 is achieved, because of the shape of the first grooves 21m and the second grooves 22m explained above, the housing space for internal components that can be built in the bending section 11 does not decrease. Therefore, performance of the internal components is not limited.

The wires 30u and 30d can be easily fit in the first grooves 21m simply by being moved from the hole 21i to the outer side in the radial direction R. The wires 30u and 30d can be easily fit in the second grooves 22m simply by being moved from the outer side to the inner side in the radial direction R, Accordingly, the wires 30u and 30d can be assembled in a shorter time more easily than a conventional method of inserting wires, from a front or a rear, through through-holes formed in thick portions of a. respective plurality of bending pieces coupled in the longitudinal axial direction N. Therefore, the bending section 11 can be inexpensively manufactured.

In the present embodiment explained above, the first bending piece 21 and the second bending piece 22 are explained as being alternately disposed in the longitudinal axial direction N.

According to the above, when the wire 30u or the wire 30d is towed in order to bend the bending section 11 in the upward direction (U) or the downward direction (D), a bending force, that is, the turning force explained above applied to the first bending piece 21 and the second bending piece 22 from the wire 30u or the wire 30d becomes uniform. Therefore, a bending shape of the bending section 11 can be stabilized.

This is because, for example, if the plurality of second bending pieces 22 equal to or more than a fixed number are continuously coupled in the longitudinal axial direction N, when the wire 30u or the wire 30d is towed to bend the bending section 11 in the upward or downward direction, the wire 30u or the wire 30d comes off from the respective second grooves 22m to the outer side in the radial direction R, the turning force in the upward direction (U) cannot be sufficiently applied to the plurality of first bending pieces 21 and the plurality of bending pieces 22 coupled in the longitudinal axial direction N, that is, the bending force applied from the wire 30u becomes nonuniform, and the bending shape of the bending section 11 becomes unstable.

This is also because, if the plurality of first bending pieces 21 equal to or more than a fixed number are continuously coupled in the longitudinal axial direction N, the wire 30u and the wire 30d easily come off to the hole 21i.

Since the wires 30u and 30d less easily move in the radial direction R from the first grooves 21m and the second grooves 22m, it is possible to minimize a load applied to the bending rubber covering the outer circumferences of the first bending piece 21 and the second bending piece 22 involved in the movement to the outer side in the radial direction R and a load applied to the internal components involved in the movement to the inner side in the radial direction R.

From the above, it is possible to provide the bending section 11 of the endoscope, the endoscope insertion section 2, and the endoscope 1 including a configuration in which it is possible to secure a housing space for internal components as large as possible while achieving a reduction in a diameter and assembly of wires is improved.

### Second Embodiment

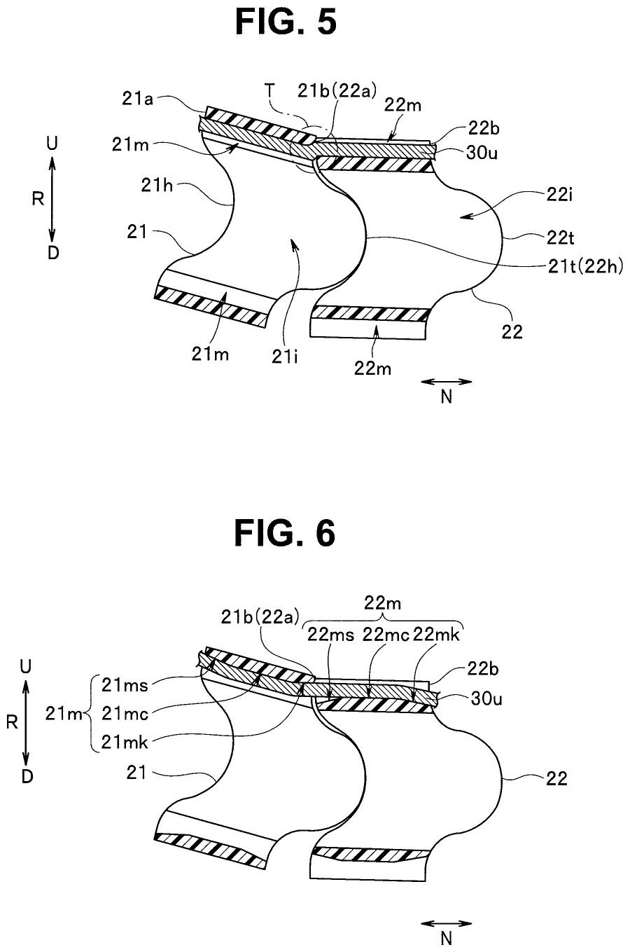

FIG. 5 is a cross-sectional view showing a state in which the first bending piece rotates in an upward direction up to a maximum rotation angle with respect to the second bending piece shown in FIG. 2. FIG. 6 is a cross-sectional view showing a state in which a first bending piece rotates in an upward direction up to a maximum rotation angle with respect to a second bending piece in a bending piece configuring a bending section of an endoscope in the present embodiment.

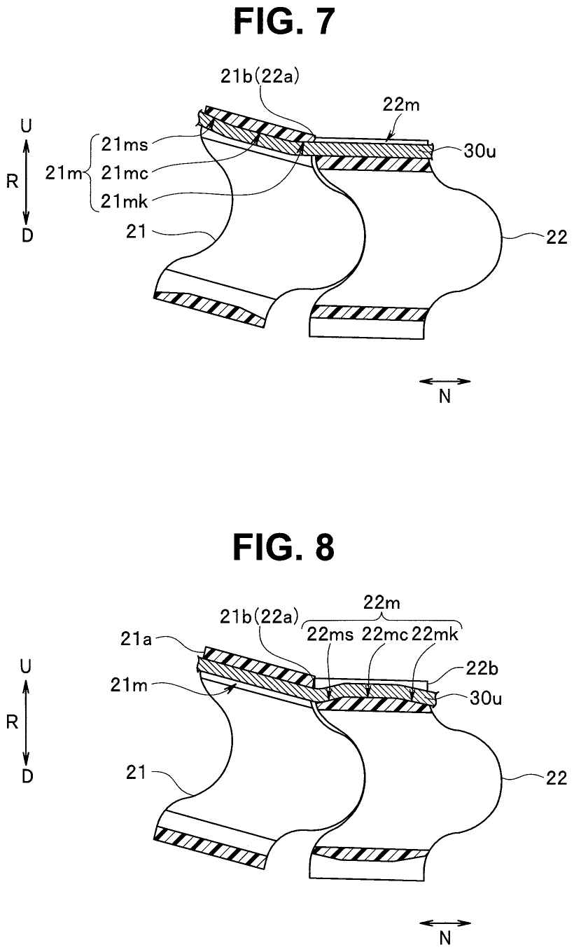

FIG. 7 is a cross-sectional view showing a modification of a shape of a second groove of the second bending piece shown in FIG. 6. FIG. 8 is a cross-sectional view showing a modification of a shape of a first groove of the first bending piece shown in FIG. 6.

Configurations of a bending section of an endoscope, an insertion section, and the endoscope in the second embodiment are different in a shape of at least one of the first groove and the second groove compared with the first embodiment shown in FIG. 1 to FIG. 4 explained above.

Accordingly, only this difference is explained. The same components as the components in the first embodiment explained above are denoted by the same reference numerals and signs and explanation of the components is omitted.

Note that, in the present embodiment, coupling of one first bending piece 21 and one second bending piece 22 is explained as an example in order to simplify the drawings and explanation. In FIG. 5 to FIG. 8, the wire 30d is omitted in order to simplify the drawings.

As shown in FIG. 3, in the first embodiment explained above, the first grooves 21m are formed with respect to the first bending piece 21 at the constant depth D in the longitudinal axial direction N from the distal end to the proximal end of the first bending piece 21.

The second grooves 22m are formed with respect to the second bending piece 22 at the constant depth D in the longitudinal axial direction N from the distal end to the proximal end of the second bending piece 22.

However, in the present configuration, as shown in FIG. 5, for example, when the wire 30u is towed, whereby the first bending piece 21 rotates with respect to the second bending piece 22 in the upward direction (U) up to a maximum rotation angle at which the shoulder portion 22a comes into contact with the shoulder portion 21b, as indicated by being surrounded by an alternate long and short dash line, in a contact section of the shoulder portion 22a and the shoulder portion 21b, a bent portion T is formed in the wire 30u. Accordingly, there is a problem in that movement of the wire 30u in the longitudinal axial direction N is prevented, the shoulder portion 22a and the shoulder portion 21b cannot rotate until coming into contact with each other, and a desired bending shape and a desired bending angle cannot be obtained for the bending section 11. The wire 30u is likely to be broken by the bent portion T.

Therefore, as shown in FIG. 6, in the present embodiment, in the first groove 21m, a distal end portion 21ms and a proximal end portion 21mk in the longitudinal axial direction N are formed deeper than an intermediate portion 21mc.

In other words, in thickness of a part where the first groove 21m is formed in the first bending piece 21, the distal end portion 21ms and the proximal end portion 21mk are formed thinner than the intermediate portion 21mc.

In other words, on a bottom surface of the first groove 21m, the distal end portion 21ms and the proximal end portion 21mk are formed further on the outer side in the radial direction R than the intermediate portion 21mc. Inclined surfaces are formed between the distal end portion 21ms and the proximal end portion 21mk and the intermediate portion 21mc.

In the second groove 22m, a distal end portion 22ms and a proximal end portion 22mk in the longitudinal axial direction N is formed deeper than an intermediate portion 22mc. In other words, in thickness of a part where the second groove 22m is formed in the second bending piece 22, the distal end portion 22ms and the proximal end portion 22mk are formed thinner than the intermediate portion 22mc.

In other words, on a bottom surface of the second groove 22m, the distal end portion 22ms and the proximal end portion 22mk are formed further on the inner side in the radial direction R than the intermediate portion 22mc. Inclined surfaces are formed between the distal end portion 22ms and the proximal end portion 22mk and the intermediate portion 22mc.

Note that, as shown in FIG. 7, the second groove 22m may be formed at the constant depth D in the longitudinal axial direction N and only the first groove 21m may be formed in the same shape as the shape shown in FIG. 6. As shown in FIG. 8, the first groove 21m may be formed at the constant depth D in the longitudinal axial direction N and only the second groove 22m may be formed in the same shape as the shape shown in FIG. 6.

Note that other components are the same as the components in the first embodiment explained above.

With such a configuration, as shown in FIG. 6 to FIG. 8, for example, the wire 30u is towed, whereby the first bending piece 21 rotates with respect to the second bending piece 22 in the upward direction (U) up to a maximum rotation angle at which the shoulder portion 22a comes into contact with the shoulder portion 21b. At this point, in a contact portion of the shoulder portion 22a and the shoulder portion 21b, because of a shape shown in FIG. 6 to FIG. 8 of the first groove 21m and the second groove 22m, the bent portion T is not formed in the wire 30u as shown in FIG. 5. Accordingly, it is possible to prevent breakage of the wire 30u.

From the above, when the bending section 11 is bent in the upward direction (U), since the wire 30u can smoothly move in the longitudinal axial direction N, the shoulder portion 22a and the shoulder portion 21b can easily rotate up to the maximum rotation angle until the shoulder portion 22a and the shoulder portion 21b come into contact with each other. Therefore, it is possible to obtain a desired bending shape and a desired bending angle for the bending section 11.

Note that other effects are the same as the effects in the first embodiment explained above.

The above is the same in the first groove 21m and the second groove 22m through which the wire 30d is inserted.

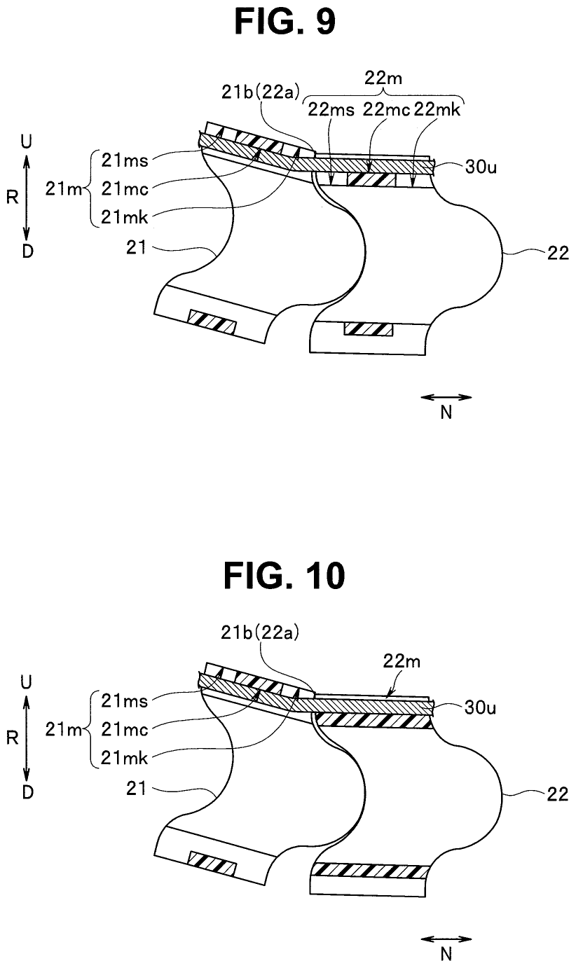

Note that modifications are explained below with reference to FIG. 9 to FIG. 11. FIG. 9 is a cross-sectional view showing a modification in which respective distal end portions and respective proximal end portions in the first groove of the first bending piece and the second groove of the second bending piece shown in FIG. 6 are formed as cutouts.

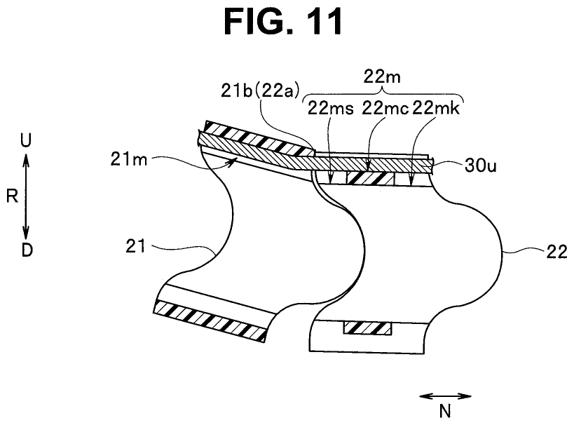

FIG. 10 is a cross-sectional view showing a modification in which respective distal end portions and respective proximal end portions in the first groove of the first bending piece and the second groove of the second bending piece shown in FIG. 7 are formed as cutouts. FIG. 11 is a cross-sectional view showing a modification in which respective distal end portions and respective proximal end portions in the first groove of the first bending piece and the second groove of the second bending piece shown in FIG. 8 are formed as cutouts.

As shown in FIG. 9 to FIG. 11, as the configuration in which the distal end portion 21ms and the proximal end portion 21mk of the first groove 21m in the first bending piece 21 are formed deeper than the intermediate portion 21mc and the configuration in which the distal end portion 22ms and the proximal end portion 22mk of the second groove 22m in the second bending piece 22 are formed deeper than the intermediate portion 22mc, a configuration in which cutouts piercing through the distal end portions 21ms and 22ms and the proximal end portions 21mk and 22mk in the radial direction R are formed in the distal end portions 21ms and 22ms and the proximal end portions 21mk and 22mk is also conceivable besides reducing the thickness of the parts where the distal end portions 21ms and 22ms and the proximal end portions 21mk and 22mk are formed in the first bending piece 21 and the second bending piece 22 shown in FIG. 6 to FIG. 8 explained below.

With such a configuration as well, it is possible to obtain the same effects as the effects in the present embodiment explained above.

### Third Embodiment

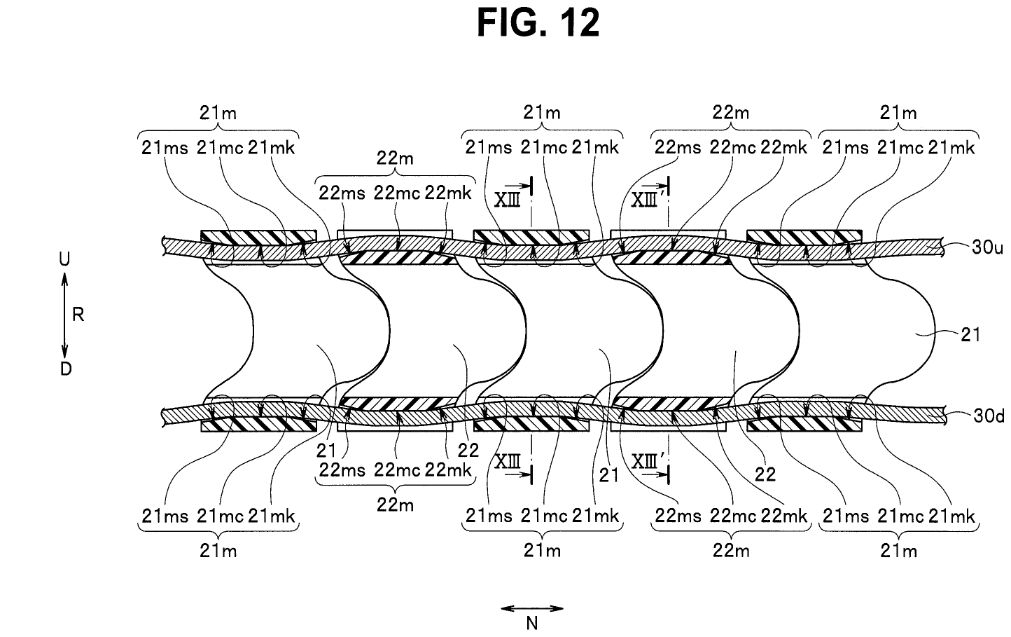

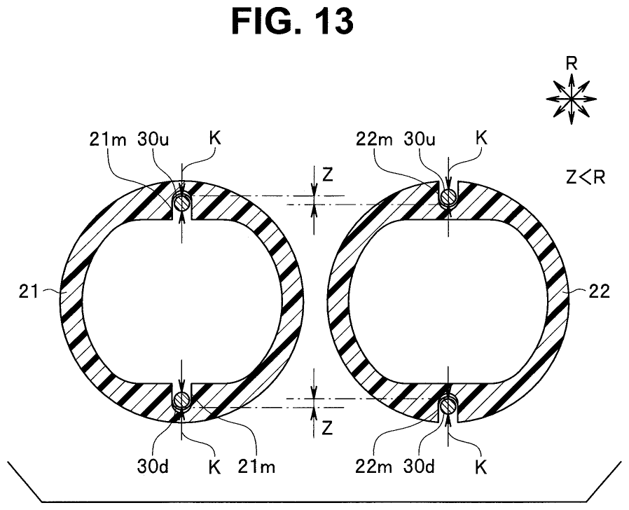

FIG. 12 is a partial cross-sectional view showing, together with two wires, a plurality of bending pieces configuring a bending section in the present embodiment. FIG. 13 is a diagram showing, together with the wires, side by side with a cross section of a second bending piece taken along a XIII′-XIII′ line in FIG. 12, a cross section of a first bending piece taken along a XIII-XIII line in FIG. 12.

Configurations of a bending section of an endoscope, an endoscope insertion section, and the endoscope in the third embodiment are different in shapes of the first groove and the second groove compared with the first embodiment shown in FIG. 1 to FIG. 4 and the second embodiment shown in FIG. 5 to FIG. 11 explained above.

Accordingly, only this difference is explained. The same components as the components in the first and second embodiments explained above are denoted by the same reference numerals and signs and explanation of the components is omitted.

As shown in FIG. 13, in the present embodiment, when the first bending piece 21 and the second bending piece 22 are coupled in the longitudinal axial direction N, bottom portions on the outer side in the radial direction R of the first grooves 21m are located further on the outer side in the radial direction R than bottom portions on the inner side in the radial direction R of the second grooves 22m.

At this time, length Z in the radial direction R between the bottom portions is smaller than the outer diameter K of the wires 30u and 30d.

Accordingly, when the wires 30u and 30d are inserted through the first grooves 21m and the second grooves 22m to collide with the respective bottom portions of the first grooves 21m and the second grooves 22m, positions in the radial direction R of the wires 30u and 30d are located further on the outer side in the radial direction R in the second grooves 22m than in the first grooves 21m.

Note that this is the same when the first groove 21m and the second groove 22m are formed at the constant depth D in the longitudinal axial direction N and when, as shown in FIG. 12, in the first groove 21m, the distal end portion 21ms and the proximal end portion 21mk are formed deeper than the intermediate portion 21mc and, in the second groove 22m, the distal end portion 22ms and the proximal end portion 22mk are formed deeper than the intermediate portion 22mc.

As a result, as shown in FIG. 12, the wires 30u and 30d inserted through the first groove 21m and the second groove 22m in the longitudinal axial direction N meander in the longitudinal axial direction N.

Note that other components are the same as the components in the first and second embodiments explained above.

With such a configuration, the wires 30u and 30d meandering in the first groove 21m and the second groove 22m are about to return to the shape extending in the longitudinal axial direction N. Therefore, since the wires 30u and 30d are pressed in the radial direction R against the respective bottom portions of the first groove 21m and the second groove 22m, the wires 30u and 30d less easily move to the outer side and the inner side in the radial direction R than the configurations in the first and second embodiments.

According to the meandering of the wires 30u and 30d, friction against the respective bottom portions of the first groove 21m and the second groove 22m increases. Therefore, when the bending section 11 is bent in the upward direction (U) or the downward direction (D), since a bending shape can be easily fixed without using a known shape lock mechanism separately provided in the operation section 3, manufacturing cost can be reduced.

Further, when performing bending operation of the bending section 11 using the bending operation lever 15, an operator can fix the bending shape of the bending section 11 even if the operator releases fingers from the bending operation lever 15. Therefore, since the operator does not need to continue to press the operated bending operation lever 15 with the fingers, it is possible to reduce a burden on the fingers.

Note that other effects are the same as the effects in the first and second embodiments explained above.

In the first to third embodiments, the bending section 11 is explained as being bendable in the up-down direction by the wires 30u and 30d. However, the bending section 11 may be configured to be bendable in the left-right direction.

In this case, the first grooves 21m are formed in two places to be respectively opposed to positions corresponding to the left-right direction in a circumferential direction of the first bending piece 21 with respect to the inner circumference 21n of the first bending piece 21. The second grooves 22m are formed in two places to be respectively opposed to positions corresponding to the left-right direction in a circumferential direction of the second bending piece 22 with respect to the outer circumference 22g of the second bending piece 22.

Further, the bending section 11 may be bendable in the up-down and left-right directions by four wires.

In this case, the first grooves 21m are formed in four places at every approximately 90° in the circumferential direction in positions corresponding to the up-down and left-right directions in the circumferential direction of, the first bending piece 21 with respect to the inner circumference 21n of the first bending piece 21. The second grooves 22m are formed in four places at every approximately 90° in the circumferential direction in positions corresponding to the up-down and left-right directions in the circumferential direction of the second bending piece 22 with respect to the outer circumference 22g of the second bending piece 22.

The first bending piece 21 and the second bending piece 22 are explained as being formed from resin in the first to third embodiments explained above but may be formed from metal. However, for example, when the first bending piece 21 and the second bending piece 22 are formed from resin and the bending section 11 is further reduced in diameter in order to inexpensively manufacture the bending section 11 used in the endoscope 1 of, for example, a disposable type, the configurations in the first to third embodiments explained above are more effective.

Further, in the first to third embodiments explained above, the first bending piece 21 and the second bending piece 22 are explained as being coupled by the rivet-less structure. However, not only this, but it goes without saying that the configurations in the first to third embodiments are applicable to the conventional rivet structure as well.

In the first to third embodiments explained above, the first bending pieces 21 and the second bending pieces 22 are explained as being alternately disposed in the longitudinal axial direction N. However, not only this, but the first bending pieces 21 and the second bending pieces 22 do not always need to be alternately disposed if the bending shape of the bending section 11 explained above does not become unstable.

For example, the first bending pieces 21 and the second bending pieces 22 may be disposed in such a manner as the first bending piece 21, the first bending piece 21, the second bending piece 22, the second bending piece 22, and the first bending piece 21 . . . in the longitudinal axial direction N.

Further, in the first to third embodiments explained above, the endoscope 1 is explained using the pyeloureteroscope as an example. However, it goes without saying that the endoscope 1 is not limited to the pyeloureteroscope. It is desirable that the endoscope 1 is applied to, in particular, an endoscope that is requested to be reduced in diameter and reduced in manufacturing cost.

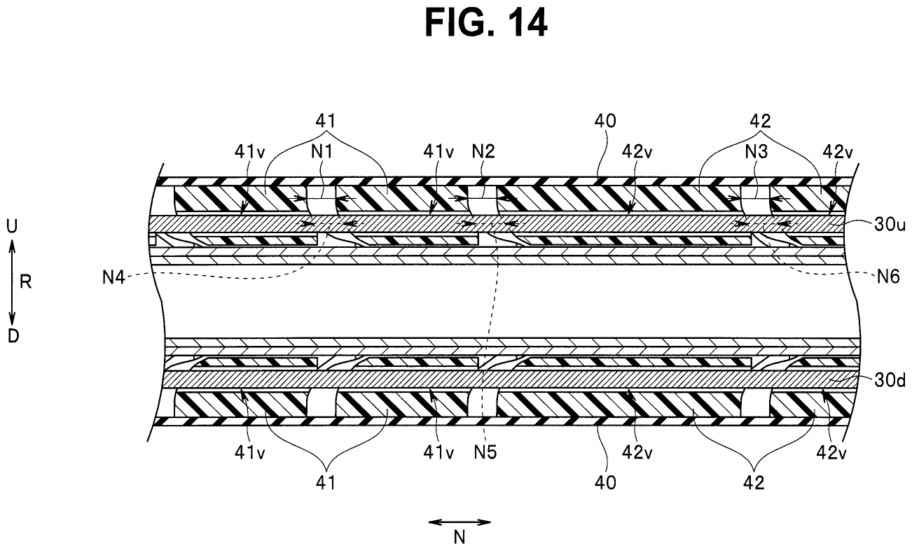

FIG. 14 is a partial cross-sectional view of a bending section of a general endoscope.

In the first to third embodiments explained above, the first grooves 21m are explained as being formed to pierce through the first bending piece 21 in the longitudinal axial direction N over the entire length from the distal end to the proximal end of the first bending piece 21. The second grooves 22m are also explained as being formed to pierce through the second bending piece 22 in the longitudinal axial direction N over the entire length from the distal end to the proximal end of the second bending piece 22.

This is also applicable to through-holes 41v and 42v formed in wire receivers of bending pieces 41 and 42 configuring the bending section of the general endoscope as shown in FIG. 14,

In other words, the through-hole 41v is formed in the wire receiver in the longitudinal axial direction N over an entire length from a distal end to a proximal end of the bending piece 41 and the through-hole 42v is formed in the wire receiver in the longitudinal axial direction N over an entire length from a distal end to a proximal end of the bending piece 42.

Accordingly, a gap distance N1 in the longitudinal axial direction N between the bending pieces 41, a gap distance N2 in the longitudinal axial direction N between the bending piece 41 and the bending piece 42, and a gap distance N3 in the longitudinal axial direction between the bending pieces 42 substantially coincide with a gap distance N4 in the longitudinal axial direction N between the through-holes 41v, a gap distance N5 in the longitudinal axial direction N between the through-hole 41v and the through-hole 42v, and a gap distance N6 between the through-holes 42v (N1=N4, N2=N5, and N3=N6).

In such a configuration, when the wires 30u and 30d are inserted through the first grooves 21m and the through-holes 41v and 42v from a front or a rear in the longitudinal axial direction N, it is easier to insert the wires 30u and 30d than a configuration in which the first grooves 21m, the second grooves 22m, and the through-holes 41v and 42v are partially formed only in a part in the longitudinal axial direction N of the first bending piece 21, the second bending piece 22, and the bending pieces 41 and 42.

If a heat shrinkable tube 40 covers outer circumferences of the bending pieces 41 and 42 as shown in FIG. 14, it is possible to effectively prevent the bending pieces 41 and 42 from deviating from each other in the radial direction R.

Note that this is also applicable When a known braid covers the outer circumferences of the bending pieces 41 and 42, In this case, the heat shrinkable tube 40 only has to cover an outer circumference of the braid.

The present configuration is also applicable in the first to third embodiments explained above. If a heat shrinkable tube covers the outer circumferences of the first bending piece 21 and the second bending piece 22, it is possible to effectively prevent the first bending piece 21 and the second bending piece 22 from deviating in the radial direction R.

A rivet-less coupling configuration of bending pieces is well known in which a semicircular (fan-shaped) convex portion formed at an end portion of the other bending piece is only caused to collide with a semicircular (fan-shaped) concave portion formed at an end portion of one bending piece between bending pieces adjacent to each other in a longitudinal axial direction of an endoscope insertion section in order to bend a bending section of an endoscope. The rivet-less coupling configuration is disclosed in U.S. Pat. No. 8,465,420, for example.

However, in some case, a force is applied to the bending section not only in the longitudinal axial direction but also in a radial direction of a longitudinal axis or a twisting force is applied to the bending section.

In this case, in the coupled bending pieces, it is likely that the other bending piece deviates in an up-down direction of the bending section in the radial direction of the longitudinal axis with respect to one bending piece, There is a problem in that it is difficult to bend the bending section in a desired bending shape.

In view of such a problem, a configuration is well known in which a known braid for deviation prevention or the like covers outer circumferences of a coupled plurality of bending pieces. However, in the present configuration, there is a problem in that an amount of bending force of the bending section increases, that is, torque followability of the bending section to a towing force of a wire for bending the bending section is deteriorated, or it is difficult to assemble the bending section.

The present configuration provides a configuration of a bending section that can prevent deviation in the up-down direction and twist of a coupled plurality of bending pieces.

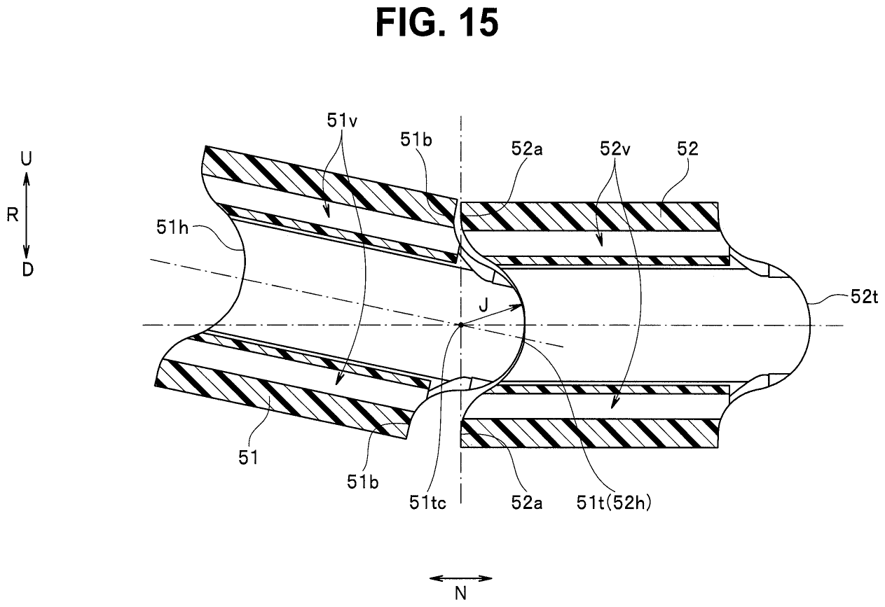

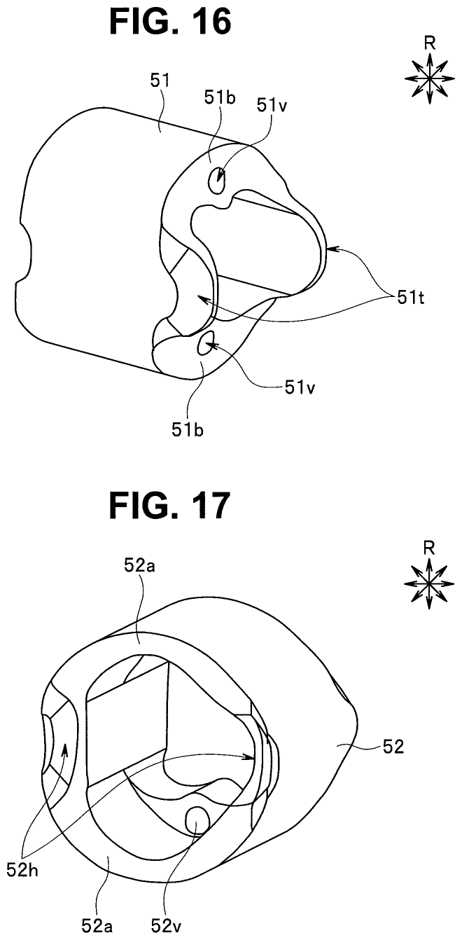

An embodiment of the present configuration is explained below with reference to FIG. 15 to FIG. 17. FIG. 15 is a partial cross-sectional view showing a plurality of bending pieces configuring a bending section of the present configuration. FIG. 16 is a perspective view of one bending piece shown in FIG. 15. FIG. 17 is a perspective view of the other bending piece shown in FIG. 15.

Note that, in FIG. 15, coupling of two bending pieces is shown as an example in order to simplify the drawing and explanation.

As shown in FIG. 15, a bending piece 51 includes, on a proximal end face. which is an end portion in the longitudinal axial direction N of an insertion section, a pair of convex portions 51t extending to the rear in the longitudinal axial direction N and symmetrical with respect to a center axis of the bending piece 51. Note that the convex portions 51t have a semicircular shape (a fan shape).

Further, the bending piece 51 includes, on a distal end face, which is an end. portion in the longitudinal axial direction N, a pair of concave portions 51h recessed to the rear in the longitudinal axial direction N and symmetrical with respect to the center axis of the bending piece 51. Note that the concave portions 51h have a semicircular shape (a fan shape).

A bending piece 52 includes, on a proximal end face, which is an end portion in the longitudinal axial direction N, a pair of convex portions 52t respectively extending to the rear in the longitudinal axial direction N and symmetrical with respect to a center axis of the bending piece 52. Note that the convex portions 52t have a semicircular shape (a fan shape).

Further, the bending piece 52 includes, on a distal end face, which is an end portion in the longitudinal axial direction N, a pair of concave portions 52h recessed to the rear in the longitudinal axial direction N and symmetrical with respect to the center axis of the bending piece 52. Note that the concave portions 52h have a semicircular shape (a fan shape).

The bending piece 51 and the bending piece 52 are disposed in the longitudinal axial direction N, whereby the pair of convex portions 51t of the bending piece 51 is in contact with the pair of concave portions 52h of the bending piece 52.

Note that the contact of the bending piece 51 and the bending piece 52 is performed in a state in which the bending piece 51 and the bending piece 52 are compressed in the longitudinal axial direction N by not-shown wires respectively inserted through through-holes 51v and 52v of the wire receivers provided in the respective bending pieces 51 and 52.

Consequently, the bending piece 51 and the bending piece 52 are configured to be turnable with respect to each other. More specifically, the bending piece 51 and the bending piece 52 are in contact to be turnable in the up-down direction (UD) in the radial direction R of the longitudinal axis N.

Accordingly, for example, when a wire located in the upward direction (U) towed to the rear, the pair of convex portions 51t in contact with the pair of concave portions 52h rotates in the upward direction (U), whereby the bending section bends in the upward direction (U) in FIG. 15.

Note that a maximum rotation angle in the upward direction (U) of the pair of convex portions 51t in contact with the pair of concave portions 52h is specified by a shoulder portion 51b at a proximal end of the bending piece 51 coming into contact with a shoulder portion 52a at a distal end of the bending piece 52.

On the contrary, when a wire located in the downward direction (D) is towed to the rear, the pair of convex portions 51t in contact with the pair of concave portions 52h rotates in the downward direction, whereby the bending section 11 bends in the downward direction (D) in FIG. 2.

Note that a maximum rotation angle in the downward direction (D) of the pair of convex portions 51t in contact with the pair of concave portions 52h is specified by the shoulder portion 51b coming into contact with the shoulder portion 52a.

In other words, the bending section in the present configuration has a known rivet-less structure in which a rivet is not used for coupling of the bending pieces 51 and 52.

Here, the convex portions 51t desirably cut in as much as possible and are contact with the concave portions 52h in order to prevent deviation in the up-down direction and twist of the coupled bending pieces 51 and 52.

However, if a cut-in amount is large, the bending piece 52 and the bending piece 51 less easily rotate in the up-down direction with respect to each other.

Therefore, as shown in FIG. 15. in the present configuration, a center point (a radial center) 51tc in the case in which a radius of a fan shape of the convex portions 51t of the bending piece 51 is represented as is configured on the same plane as the shoulder portion 52a of the bending piece 52 in the radial direction R. Note that the center point 51tc is located on the same plane as a center axis in the longitudinal axial direction of the bending pieces 51 and 52.

Note that, although not shown, a radial center of the convex portions 52t of the bending piece 52 is configured the same.

The configuration explained above is also applicable in a configuration in which the convex portions 52t are provided at the distal end of the bending piece 52 and are in contact with the concave portions 51h provided at the proximal end of the bending piece 51.

With such a configuration, since the convex portions 52t cut in the concave portions 51h at an appropriate contact amount and are in contact with the concave portions 51h without excessively cutting in, it is possible to surely prevent the coupled bending pieces 51 and 52 from deviating in the up-down direction or twisting. Therefore, bending of the bending section is not hindered.

Accordingly, it is possible to secure torque followability of the bending section to a towing force of the wires and cause the bending section to bend in a desired bending shape. Note that the present configuration is also applicable in the first to third embodiments explained above.

In FIG. 15, the convex portions 51t, the concave portions 51h, the convex portions 52t, and the concave portions 52h are shown as being formed in the semicircular shape (the fan shape).

Not only this, but, as shown in FIG. 16 and FIG. 17, the convex portions 51t may be formed in a conical shape in which surfaces of the convex portions 51t in contact with the concave portions 52h are not surfaces substantially perpendicular to longitudinal axes of the bending pieces 51 and 52 but are obliquely in contact with the concave portions 52h. The concave portions 52h may be formed in a conical shape in which surfaces of the concave portions 52h in contact with the convex portions 51t are obliquely in contact with the convex portions 51t according to the shape of the convex portions 51t.

Note that, in this case, an outer diameter of the concave portions 52h and 51h is desirably formed larger than an outer diameter of the convex portions 51t and 52t.

Although not shown, the convex portions 52t and the concave portions 51h may be formed in the same shapes as the shapes of the convex portions 51t and the concave portions 52h.

With such a configuration, since the convex portions 51t are obliquely in contact with the concave portions 52h, the convex portions 51t more easily cut in the concave portions 52h. Therefore, it is possible to effectively suppress deviation in the up-down direction of the bending pieces 51 and 52.

Note that the present configuration is also applicable to the first to third embodiments explained above.

A configuration is well known in which an endoscope bending section includes an active bending section that actively bends and a passive bending section that is consecutively connected to a proximal end side of the bending section and passively bends. The configuration is disclosed in Japanese Patent Application Laid-Open Publication No. 2016-174670.

Here, in a plurality of bending pieces configuring the active bending section, in general, a wire through-hole of a wire receiver provided in a proximal end bending piece that is located closest to a proximal end side in a longitudinal axial direction of the endoscope insertion section and to which a distal end of the passive bending section is connected is disposed further on an inner side in a radial direction in a longitudinal axis of an insertion section than a wire through-hole of a wire receiver of another bending piece.

This is because it is desired to insert the wires through on the radial direction inner side as much as possible in the passive bending section as well in order to achieve a reduction in a diameter of the passive bending section.

However, if a wire through-hole of the proximal end bending piece is located further on the inner side in the radial direction than a wire through-hole of a bending piece immediately preceding the proximal bending piece, when the wires are inserted through the wire through-holes of the respective bending pieces, it is likely that the wires greatly meander between the proximal end bending piece and the immediately preceding bending piece and a bend occurs in the wires.

As a result, there is a problem in that, when the wires are towed, noise occurs or an amount of towing force of the wires increases.

In view of the problems described above, the present configuration provides a. configuration of a bending section of an endoscope having an object of reducing meandering of wires between a bending piece located at a proximal end of an active bending section and a bending piece immediately preceding the bending piece.

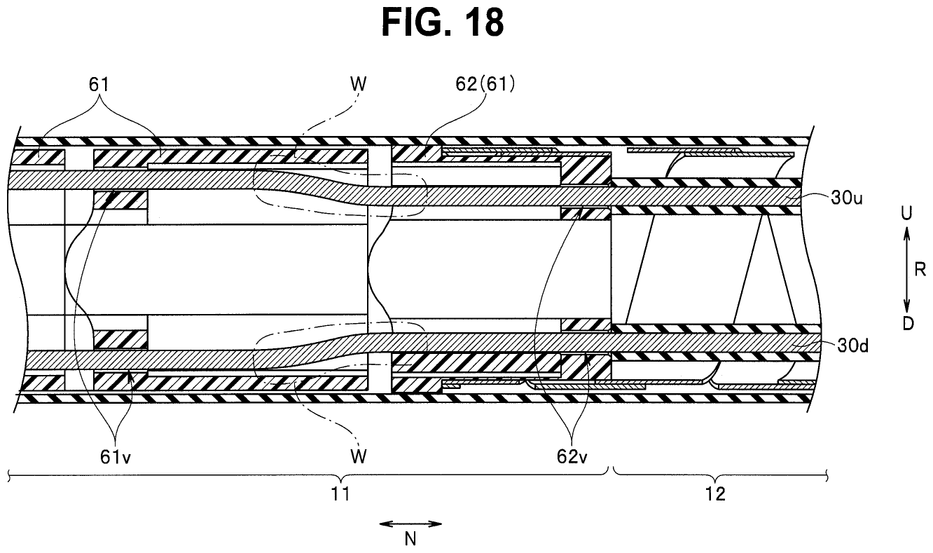

The configuration for solving the problems described above is explained below with reference to FIG. 18. FIG. 18 is a partial cross-sectional view in a coupling part of an active bending section and a passive bending section in a bending section of the present configuration.

As shown in FIG. 18, in the present configuration, the bending section of the endoscope includes the active bending section (hereinafter simply referred to as bending section) 11 and the passive bending section 12.

The bending section 11 is configured to be bendable in, for example, upward and downward two directions according to towing of the wires 30u and 30d. Note that the bending section 11 may be configured to be bendable in left and right two directions or may be configured to be bendable in upward, downward, left, and right four directions.

The bending section 11 is configured by a plurality of bending pieces 61 being coupled in the longitudinal axial direction N of an insertion section. Note that a reference numeral 62 is added to a bending piece located closest to a proximal end side in the coupled plurality of bending pieces.

The passive bending section 12 is configured from a known flex or the like and configured softly to be able to passively bend according to an external force.

Here, in the bending section H, through-holes 61v of wire receivers and through-holes 62v of wire receivers through which the wires 30u and 30d are inserted are formed in the respective bending pieces 61 and 62.

In the present configuration, wire receivers are formed on distal end sides of the respective bending pieces 61. The through-holes 61v are formed in the respective wire receivers. Wire receivers are also formed on proximal end sides of the respective bending pieces 62. The through-holes 62v are formed in the wire receivers.

With such a configuration, the meandering of the wires 30u and 30d explained above that occurs in a space W between the bending piece 62 and the bending piece 61 in the longitudinal axial direction N can be reduced because the through-holes 62v and the through-holes 61v of the bending piece 61 immediately preceding the bending piece 62 are greatly separated and disposed in the longitudinal axial direction N.

Note that the configuration explained above is also applicable in the first to third embodiments explained above.

It goes without saying that the configuration is also applicable in a configuration in which the passive bending section 12 is not provided in the endoscope insertion section and a known flexible tube section is directly consecutively connected to the proximal end of the bending section.

In the conventional endoscope, various internal units such as an image pickup unit, an illumination unit, and a treatment instrument channel are disposed inside a distal end portion of the insertion section. These various internal units are respectively fixed in predetermined positions using, for example, an adhesive with respect to a distal end rigid member configuring an endoscope distal end portion.

Conventionally, in a manufacturing process for the endoscope distal end portion, work for accurately incorporating components and constituent units of each of these various internal units in predetermined positions inside the distal end rigid member using, for example, a jig has been performed. High accuracy is requested for the incorporating work.

More specifically, for example, in order to dispose an illumination unit including an illumination lens and a light guide cable in a predetermined position inside the distal end rigid member, first, an extremely small illumination lens is disposed in a predetermined position near a distal end face of the distal end rigid member. Thereafter, a distal end of the light guide cable is disposed in a predetermined position in contact with the illumination unit incorporated in the distal end rigid member.

In this case, a relative positional relation between the illumination lens and the distal end of the light guide cable is strictly specified. Accordingly, a highly accurate incorporating technique is requested even for incorporating the illumination unit.

Because of the above description, the work for highly accurately incorporating the various internal units in the distal end rigid member of the endoscope distal end portion is work requiring time. This is a cause of an increase in manufacturing cost of the endoscope.

In recent years, since a single-use endoscope has started to be spread as an endoscope, there has always been a request for a reduction in manufacturing cost. In particular, there has been a demand for reducing manufacturing cost by realizing simplification of an assembly process while securing accuracy of incorporating work.

There has been also a demand fir stably performing work for accurately disposing, in a predetermined position inside the distal end rigid member, the distal end of the light guide cable with respect to the illumination lens incorporated in the distal end rigid member.

It is convenient if an incorporated state of the respective components and constituent units can be checked, for example, immediately after the assembly work or during the work as a device for the above.

The present configuration has been made in view of the point described above and an object of the present configuration is to provide an endoscope distal end portion including a structure that can highly accurately secure stable incorporating accuracy while simplifying a work process in incorporating, in a distal end rigid member, various internal units disposed inside the endoscope distal end portion.

In order to achieve the object, an endoscope distal end portion in an aspect of the present configuration includes a. distal end rigid member including: a distal end cover member that is formed in a substantially tubular shape and covers an outer surface of the endoscope distal end portion; and a distal end base member loaded inside the distal end cover member in a state in which a plurality of internal units incorporated in the endoscope distal end portion are fixed. The distal end base member includes a cutout section for exposing, to an outside, a part of each of the plurality of internal units when the plurality of internal units are fixed in respective predetermined positions.

According to the present configuration, it is possible to provide an endoscope distal end portion including a structure that can highly accurately secure stable incorporating accuracy while simplifying a work process in incorporating, in a distal end rigid member, various internal units disposed inside the endoscope distal end portion.

### Fourth Embodiment

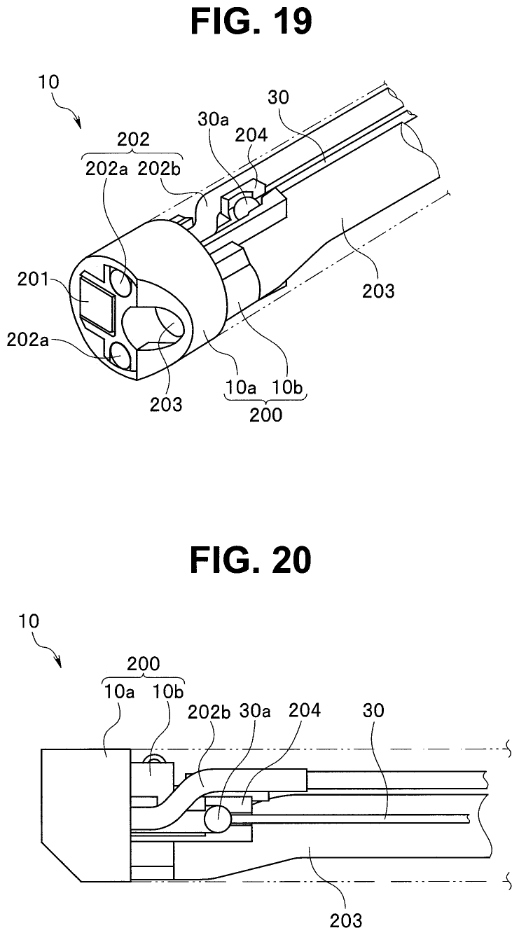

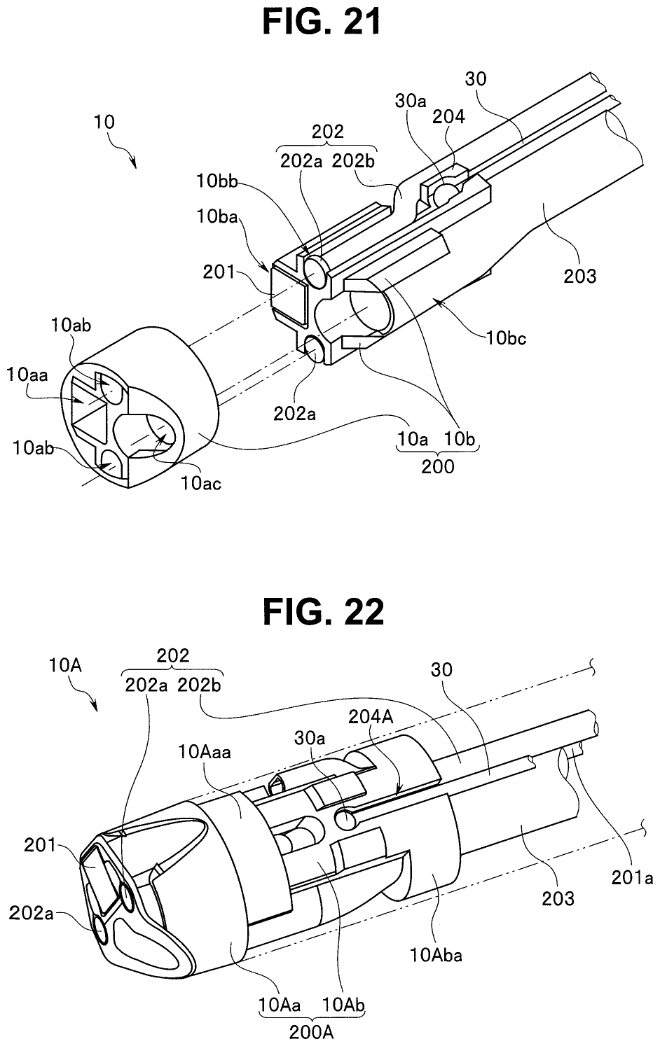

FIG. 19 to FIG. 21 are diagrams showing a fourth embodiment of the present configuration, Among the figures, FIG. 19 is a main part enlarged perspective view enlarging and showing a schematic configuration of an endoscope distal end portion in the present embodiment. FIG. 20 is a side view of the endoscope distal end portion shown in FIG. 19. FIG. 21 is an exploded perspective view showing a state in which a distal end cover is removed at the endoscope distal end portion shown in FIG. 19.

At a distal end portion 10 of an endoscope in the present configuration, as shown in FIG. 19 to FIG. 21, a distal end rigid member 200, which is a distal end frame member that incorporates and fixes various internal units such as an image pickup unit 201, an illumination unit 202, and a treatment instrument channel 203, is disposed. In the present embodiment, the distal end rigid member 200 is configured as a doubled structure of a distal end cover member 10a and a distal end base member 10b.

The distal end cover member 10a is a frame component that is formed in a substantially columnar shape and covers outer surfaces of the various internal units when the distal end base member 10b is loaded on an inner side of the distal end cover member 10a. As shown in FIG. 21, an observation window 10aa, an illumination window 10ab, and a channel opening 10ac are formed on a distal end face of the distal end cover member 10a. Note that, in the present embodiment, an example in which two illumination windows 10ab are provided is explained. In this case, the two illumination windows 10ab are disposed to sandwich the observation window 10aa near a periphery of the observation window 10aa.

The distal end base member 10b is a base member that fixes and integrates parts of respective distal end portion vicinities of a plurality of internal units (201, 202, and 203) incorporated in the distal end portion 10. After fixing and integrating the plurality of internal units (201, 202, and 203), the distal end base member 10b is loaded on the inner side of the distal end cover member 10a. The distal end base member 10b is, for example, bonded and fixed in a state in which the distal end base member 10b is loaded in a predetermined position on the inner side of the distal end cover member 10a.

Here, the image pickup unit 201 is configured by an image pickup device such as a CCD or a CMOS, an image pickup substrate on which, for example, a driving circuit for driving the image pickup device is mounted, an image pickup signal cable extending from the image pickup substrate, and the like,

The illumination unit 202 mainly includes a lens for illumination 202a and a light guide cable 202b. The lens for illumination 202a is an optical lens provided to obtain predetermined light distribution when illumination light guided from a not-shown light source apparatus by the light guide cable 202b is irradiated toward a front of the distal end portion 10. The lens for illumination 202a is fixed to a predetermined position of the distal end base member 10b. When the distal end base member 10b is loaded in the distal end cover member 10a, the lens for illumination 202a is disposed in the illumination window 10ab of the distal end cover member 10a. The light guide cable 202b is an optical fiber cable for guiding, to the distal end portion 10, illumination light emitted from the not-shown light source apparatus. The light guide cable 202b is fixed in a predetermined position of the distal end base member 10b in a state in which a distal end face of the light guide cable 202b is disposed in a position opposed to the lens for illumination 202a.

The treatment instrument channel 203 is a tubular member inserted through and disposed inside the insertion section of the endoscope in a state in which one end of the tubular member is consecutively connected to a treatment instrument insertion opening (not shown) of an operation section (not shown in FIG. 19 and the like) in the endoscope and the other end of the tubular member is fixed in a predetermined position of the distal end base member 10b of the distal end portion 10 in the endoscope. Various predetermined treatment instruments inserted from the treatment instrument insertion opening are inserted through the treatment instrument channel 203. Distal ends of the treatment instruments inserted through the treatment instrument channel 203 project forward from a channel opening 10ac on a front surface of the distal end cover member 10a of the distal end portion 10.

Note that these plurality of internal units have substantially the same components as the components of the internal units having general components applied in the conventional endoscope, Therefore, further explanation and illustration of the internal units are omitted.

In the distal end base member 10b, a plurality of receiving sections (10ba, 10bb, and 10bc) for disposing and fixing the parts of the respective distal end portion vicinities of the various internal units (201, 202, and 203) are formed. In other words, there are the receiving section 10ba in which the image pickup unit 201 is disposed, the receiving section 10bb in which the illumination unit 202 is disposed, and the receiving section 10bc in which the treatment instrument channel 203 is disposed. Note that, in a configuration example in the present embodiment, two illumination units 202 are disposed. Therefore, two receiving sections 10bb corresponding to the illumination units 202 are provided. The two receiving sections 10bb are disposed to sandwich the receiving section 10ba corresponding to the image pickup unit 201. In these plurality of receiving sections (10ba, 10bb, and 10bc), cutout sections for exposing parts of the respective internal units to the outside when the respective predetermined internal units are respectively fixed to the plurality of receiving sections (10ba, 10bb, and 10bc) are formed.

A wire fixing section 204 that fixes a distal end of a bending wire 30 is formed in a predetermined part of the distal end base member 10b. In this case, the wire fixing section 204 is formed to include a groove through which the bending wire 30 is inserted and a housing chamber that houses a spherical locking member 30a fixed to the distal end of the bending wire 30. Note that, in the present embodiment, a configuration example is explained in which the wire fixing section 204 is provided on an outer circumferential surface closer to a proximal end of the distal end base member 10b.

Here, the bending wire 30 is a wire for towing for bending a direction in which a distal end face of a. distal end portion of the insertion section of the endoscope faces to, for example, left and right or upward and downward two directions or upward, downward, left, and right four directions, Therefore, two or four bending wires 30 are disposed. In the present embodiment, an example is explained in which two bending wires 30 are provided. However, in FIG. 19 to FIG. 21, only one bending wire 30 is shown. The other one is disposed in a hidden position.

Note that the distal end cover member 10a and the distal end base member 10b are formed using, for example, a resin material having an insulation property.

The endoscope distal end portion 10 configured as explained above is generally assembled as explained below. First, the various internal units (201, 202, and 203) are incorporated in the distal end base member 10b respectively in predetermined positions and in predetermined forms.

In other words, in the receiving section 10ba of the distal end base member 10b, the image pickup unit 201 is disposed and bonded using, for example, an ultraviolet curing adhesive. At this time, for example, a light receiving surface of the image pickup unit 201 is disposed to be flush with a distal end face of the receiving section 10ba.

Similarly, in the two receiving sections 10bb of the distal end base member 10b, respective distal ends of the two illumination units 202 are disposed and bonded using, for example, an ultraviolet curing adhesive. In this case, first, the lenses for illumination 202a are disposed and bonded in distal end portions of the respective receiving sections 10bb of the distal end base member 10b. At this time, optical axes (not shown) of the lenses for illumination 202a are disposed to be substantially orthogonal to a distal end face of the receiving section 10bb. Subsequently, a distal end face of the light guide cable 202b is disposed and bonded to be opposed to the lenses for illumination 202a already fixed in predetermined positions of the distal end base member 10b. At this time, the optical axes of the lenses for illumination 202a and an illumination optical axis of the light guide cable 202b are disposed to substantially coincide.

Similarly, in the receiving section 10bb of the distal end base member 10b, a distal end of the treatment instrument channel 203 is disposed and bonded using, for example, an ultraviolet curing adhesive.

When the various internal units (201, 202, and 203) are respectively disposed with respect to the respective predetermined receiving sections (10ba, 10bb, and 10bc) of the distal end base member 10b, a part of each of the respective internal units (201, 202, and 203) is exposed to the outside from the cutout section. Accordingly, the operator can easily check, during work, whether each of the respective internal units (201, 202, and 203) is disposed in the predetermined position in the distal end base member 10b in the predetermined state.

In this case, in particular, a relative positional relation between the lenses for illumination 202a and the light guide cable 202b needs to be accurately set. Therefore, it is convenient in terms of work efficiency as well that the positional relation can be checked during the work.

Therefore, during work for assembling the respective internal units (201, 202, and 203) to the distal end base member 10b, since the operator can proceed with the work while checking an assembled state, the operator can always perform reliable and accurate assembly work.

Subsequently, the distal end base member 10b in which the respective internal units (201, 202 and 203) are assembled and integrated is loaded in a predetermined position on an inner side of the distal end cover member 10a and bonded using, for example, an ultraviolet curing adhesive. Consequently, the distal end cover member 10a and the distal end base member 10b are assembled as the distal end rigid member 200 in a state in which the distal end cover member 10a. and the distal end base member 10b are integrated.

As explained above, according to the fourth embodiment, the distal end rigid member 200 is configured by two components of the distal end cover member 10a and the distal end base member 10b and the various internal units (201, 202, and 203) are assembled to the distal end base member 10b in advance, and, thereafter, the integrated distal end base member 10b is assembled to the distal end cover member 10a. Therefore, it is possible to realize simplification of complicated work and contribute to improvement of work efficiency while securing assembly accuracy,

During work for assembling the various internal units (201, 202, and 203) to the distal end base member 10b, the operator can proceed with the work while checking assembled states of the respective units. Therefore, it is possible to always secure reliable and accurate assembly work. Accordingly, it is possible to always maintain highly accurate assembly and contribute to improvement of yield.

### Fifth Embodiment

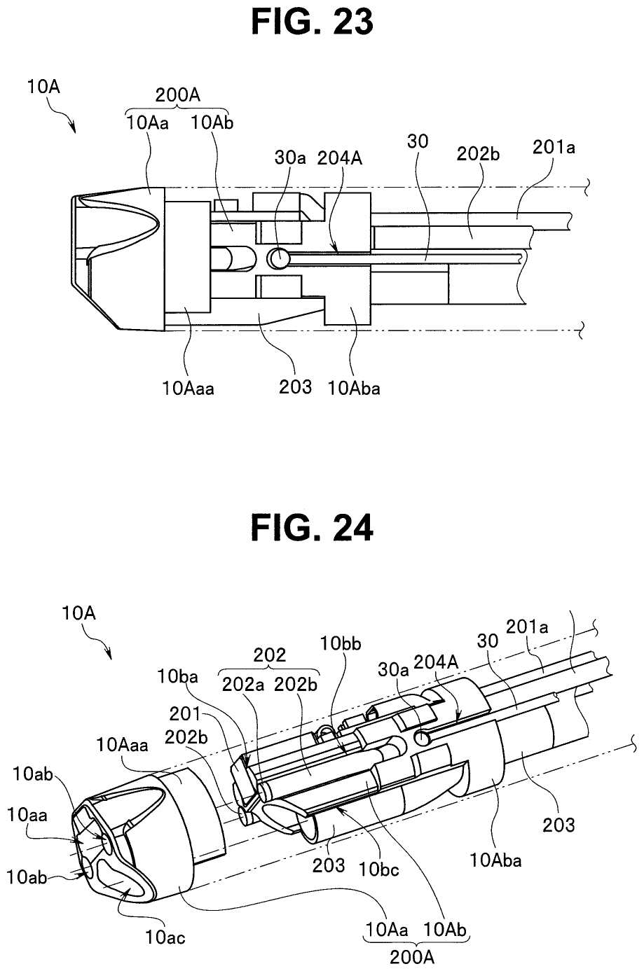

FIG. 22 to FIG. 24 are diagrams showing a fifth embodiment of the present configuration. Among the figures, FIG. 22 is a main part enlarged perspective view enlarging and showing a schematic configuration of an endoscope distal end portion in the present embodiment. FIG. 23 is a side view of the endoscope distal end portion shown in FIG. 22. FIG. 24 is an exploded perspective view showing a state in which a distal end cover is removed at the endoscope distal end portion shown in FIG. 22.

A basic configuration in the present embodiment is substantially the same as the basic configuration in the fourth embodiment explained above. In the present embodiment, a configuration of a distal end base member 10Ab is configured to be slightly differentiated. Therefore, the same components as the components in the fourth embodiment are denoted by the same reference numerals and signs and explanation of the components is omitted. Only differences are explained below.

At the distal end portion 10 of the endoscope in the fourth embodiment explained above, when the various internal units (201, 202, and 203) are respectively disposed and fixed to the respective receiving sections (10baa, 10bb, and 10bc) of the distal end base member 10b, parts of the respective distal end portion vicinities of the various internal units are fixed.

On the other hand, at a distal end portion 10A of an endoscope in the present embodiment, configurations of a distal end cover member 10Aa and a cable holding section 10Aba configuring a distal end rigid member 200A are slightly different.

In other words, the distal end cover member 10Aa is formed such that a part of the distal end cover member 10Aa projects from a proximal end face toward a proximal end side and is formed by providing a protection wall 10Aaa that covers and protects a part of an outer circumferential surface. The protection wall 10Aaa is formed to mainly cover a side surface of the illumination unit 202. Therefore, the protection wall 10Aaa has a function of blocking unnecessary light leaking from the light guide cable 202b of the illumination unit 202 toward a side direction and the like.

The distal end base member 10Ab is configured by providing the cable holding section 10Aba on a proximal end side in addition to a configuration for bonding and fixing a part of a distal end portion vicinity (the same configuration as the configuration in the fourth embodiment). The cable holding section 10Aba is a constituent section that holds, in a bound form, various cables (for example, the image pickup signal cable 201a and the light guide cable 202b) extending from the various internal units (201 and 202) toward the rear (the proximal end side) and the treatment instrument channel 203. The cable holding section 10Aba is integrally formed in a part closer to a proximal end of the distal end base member 10Ab and is formed in, for example, an annular shape.

A wire fixing section 204A that fixes the distal end of the bending wire 30 is formed on an outer circumference surface side of the cable holding section 10Aba. As in the fourth embodiment, the wire fixing section 204A includes a groove through which the bending wire 30 is inserted and a housing chamber that houses the distal end spherical locking member 30a of the bending wire 30. In the present embodiment, the wire fixing section 204A is different in that the wire fixing section 204A is formed in a form in which the cable holding section 10Aba of the distal end base member 10Ab is cut out and is formed to be disposed in a form in which the bending wire 30 and the distal end spherical locking member 30a are embedded in the cable holding section 10Aba. The other components are the same as the components in the fourth embodiment explained above.

An assembly procedure for the endoscope distal end portion 10A in the present embodiment is also substantially the same as the assembly procedure in the fourth embodiment explained above.

Note that, in the present embodiment, when the various internal units (201, 202, and 203) are incorporated in the distal end base member 10Ab, first, the image pickup signal cable 201a extending from the image pickup unit 201, the light guide cable 202b extending from the illumination unit 202, and the treatment instrument channel 203 are inserted through from a proximal end side toward a distal end side of the cable holding section 10Aba. Thereafter, parts of the distal end portion vicinities of the various internal units (201, 202, and 203) are bonded and fixed. The following incorporating work procedure is the same as the incorporating work procedure in the fourth embodiment explained above.

As explained above, according to the fifth embodiment, the same effects as the effects in the fourth embodiment explained above can be obtained. In addition, in the present embodiment, since the protection wall 10Aaa is provided in the distal end cover member 10Aa, unnecessary light leaking from the light guide cable 202b can be blocked. Accordingly, an adverse effect to video data acquired by the image pickup unit 201 can be prevented.

In the present embodiment, since the cable holding section 10Aba is provided in the distal end base member 10Ab, the image pickup signal cable 201a and the light guide cable 202b extending to the rear from the various internal units and the treatment instrument channel 203 can be held in a bound form. With the present configuration, the various cables (201a and 202b) and the treatment instrument channel 203 are bonded and fixed on a distal end side and are held by the cable holding section 10Aba on a proximal end side. Accordingly, the various cables (201a and 202b) and the treatment instrument channel 203 can be fixed to the distal end base member 10Ab (the distal end rigid member 200A) in a more stable form.

Further, since the wire fixing section 204A is configured to dispose the bending wire 30 and the distal end spherical locking member 30a in the cable holding section 10Aba of the distal end base member 10Ab in an embedded form, projection of a wire fixing portion in a radial direction can be prevented. Therefore, it is possible to contribute to a reduction in a diameter of the distal end rigid member 200A.

In a conventional endoscope, various internal units such as an image pickup unit, an illumination unit, and a treatment instrument channel are disposed inside a distal end portion of an insertion section. These various internal units are respectively fixed in predetermined positions using, for example, an adhesive with respect to a distal end rigid member configuring an endoscope distal end portion.

Among the internal units, the illumination unit is configured by an illumination lens, a light guide cable, and the like in a general configuration. Here, the light guide cable is a constituent member that is inserted through insides of an operation section and an endoscope insertion section and extended to the endoscope distal end portion from a universal cable and guides illumination light emitted from a light source apparatus, to which a universal cable connector is connected, to the endoscope distal end portion. The illumination light guided to the endoscope distal end portion in this way is emitted from a front surface window of the endoscope distal end portion toward a front of the endoscope. In this case, a configuration for distributing the illumination light to a wide range in the front of the endoscope is desirable.

Therefore, as the conventional general endoscope, there is an endoscope configured by providing, in an endoscope distal end portion, an optical lens having a predetermined shape (for example, a concave shape or a convex shape) in a position opposed to a distalmost end face of the light guide cable, for example, in a front surface window of the endoscope distal end portion in order to diffuse and distribute the illumination light emitted from the distal end of the light guide cable to a wider range.

However, when the optical lens having the form explained above is used in order to diffuse the illumination light, in addition to an increase in the number of components, since assembly work is complicated, there is a problem in that manufacturing cost is increased.

Therefore, as the conventional endoscope, an endoscope is conceived in which a predetermined part (that is, a position opposed to a distal end face of the light guide cable) of, for example, a member for fixing a light guide cable, for example, a distal end rigid member is integrated in a form in which a function of an optical lens is imparted to the member.

When such a configuration is adopted, the member (the distal end rigid member) itself for fixing the light guide cable needs to be formed of a transparent material. Accordingly, it is likely that illumination light emitted from the light guide cable enters an unintended direction, for example, an image pickup surface of the image pickup unit. In this case, a problem occurs in that the image pickup unit cannot acquire a normal image. Accordingly, in the conventional endoscope of this type, for example, a light blocking member for preventing intrusion of unnecessary light into the image pickup unit is necessary. Therefore, in this case as well, since the number of components increases, there is a problem of complication of a manufacturing process and an increase in manufacturing cost.

Therefore, in the conventional endoscope, various proposals concerning a configuration for devising a distal end shape of the light guide cable and distributing the illumination light to a wide range have been made by, for example, Japanese Patent Application Laid-Open Publication No. H09-80324.

In an endoscope disclosed by Japanese Patent Application Laid-Open Publication No. H09-80324 described above, a plurality of optical fibers are disposed around an image pickup unit and outward taper surfaces are provided on distal end faces of the respective optical fibers to obtain a large irradiation angle and realize widening of a range of the light distribution.

In the endoscope disclosed by Japanese Patent Application Laid-Open Publication No. H09-80324 described above, emitted light from the outward taper surfaces provided on the distal end faces of the respective optical fibers can contribute to expansion of the light distribution in an outward direction and incidence of unnecessary light on a light receiving surface of the image pickup unit disposed to be surrounded by the plurality of optical fibers can be avoided.

However, in terms of light amount control for illumination light for an observation target object on a front surface of the endoscope (in particular, an image pickup surface (a light receiving surface) of the image pickup unit), a case is conceivable in which sufficient brightness cannot be secured and, therefore, an object image of the observation target object formed on the light receiving surface of the image pickup unit cannot be illuminated with sufficient brightness.

The present configuration has been made in view of the above point and an object of the present configuration is to provide an endoscope distal end portion including a structure that can irradiate an observation target object on a front surface of an endoscope with sufficient illumination light and can realize distribution of the illumination light to a wider range without involving an increase in the number of components.

In order to achieve the above object, an endoscope distal end portion according to an aspect of the present configuration includes an image pickup unit, a plurality of light guides, and a distal end rigid member that fixes and holds the image pickup unit and distal ends of the light guides. A distal end shape of the plurality of light guides are formed in a convex spherical shape directed to a front or a concave shape directed to a rear.

With the present configuration, it is possible to provide an endoscope distal end portion including a structure that can irradiate an observation target object on a front surface of an endoscope with sufficient illumination light and can realize distribution of the illumination light to a wider range without involving an increase in the number of components.

### Sixth Embodiment

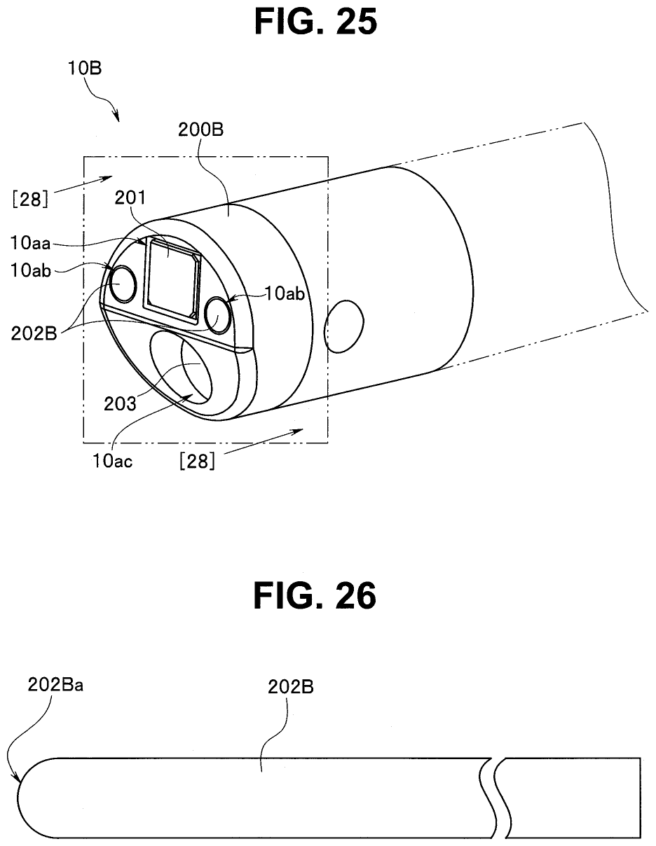

FIG. 25 to FIG. 27 are diagrams showing a sixth embodiment of the present configuration, Among the figures, FIG. 25 is a main part enlarged exterior perspective view showing an exterior of an endoscope distal end portion in the present embodiment. FIG. 26 is a side view showing a vicinity of a distal end portion of a light guide cable in a form applied to the endoscope distal end portion in the present embodiment. FIG. 27 is a side view showing a vicinity of a distal end portion of a light guide cable in another form applied to the endoscope distal end portion in the present embodiment.

First, a schematic configuration of a distal end portion 10B of an endoscope of the present configuration is explained. At the distal end portion 10B, as shown in FIG. 25. for example, a distal end rigid member 200B, which is a distal end frame member that incorporates and fixes various internal units such as the image pickup unit 201, a light guide cable 202B, which is an illumination unit, and the treatment instrument channel 203, is disposed,

The distal end rigid member 200B is a frame component that is formed in a substantially columnar shape and in which the various internal units are disposed. As shown in FIG. 25, an observation window 10aa, an illumination window 10ab, and a channel opening 10ac are formed on a distal end face of the distal end rigid member 200B. Note that, in the present embodiment, an example in which two illumination windows 10ab are provided is explained. In this case, the two illumination windows 10ab are disposed to sandwich the observation window 10aa near a periphery of the observation window 10aa.

The image pickup unit 201 is bonded and fixed inside the observation window 10aa. A distal end portion of the light guide cable 202B is bonded and fixed inside the illumination window 10ab. Further, a distal end portion of the treatment instrument channel 203 is bonded and fixed inside the channel opening 10ac.

The image pickup unit 201 includes an image pickup device such as a CCD or a CMOS, an image pickup substrate on which, for example, a driving circuit for driving the image pickup device is mounted, an image pickup signal cable extending from the image pickup substrate, and the like.

In the present embodiment, the illumination unit is configured using the light guide cable 202B. The light guide cable 202B is formed such that, as shown in FIG. 26, a shape of a distal end portion is a convex spherical shape (hereinafter simply referred to as convex shape) 202Ba directed to the front. Note that “front” in this case indicates a distal end side (a side closer to a distal end portion) of an insertion section in the endoscope. The light guide cable 202B is an optical fiber cable for guiding, to the distal end portion 10B, illumination light emitted from a not-shown light source apparatus.

As explained above, the distal end portion of the light guide cable 202B is formed in the convex shape 202Ba. By forming the distal end portion in such a shape, it is possible to obtain desired light distribution when the illumination light guided from the not-shown light source apparatus by the light guide cable 202B is irradiated toward the front of the distal end portion 10B.

In other words, in the present embodiment, predetermined front surface light distribution can be obtained by forming, instead of the lens for illumination used for the front surface light distribution in the conventional illumination unit, the distal end portion of the light guide cable 202B to be the convex shape 202Ba.

The treatment instrument channel 203 is a tubular member inserted through and disposed inside the insertion section of the endoscope in a state in which one end of the tubular member is consecutively connected to a treatment instrument insertion opening (not shown) of an operation section (not shown) in the endoscope and the other end of the tubular member is fixed in a predetermined position inside the distal end rigid member 200B of the distal end portion 10B in the endoscope. Various predetermined treatment instruments inserted from the treatment instrument insertion opening are inserted through the treatment instrument channel 203. Distal ends of the treatment instruments inserted through the treatment instrument channel 203 project forward from the channel opening 10ac on a front surface of the distal end rigid member 200B of the distal end portion 10B.

Note that the distal end portion of the light guide cable 202B configuring the illumination unit is bonded and fixed to a predetermined position inside the distal end rigid member 200B. In the present embodiment, as shown in FIG. 25, two light guide cables 202B are provided. The two light guide cables 202B are disposed to sandwich the image pickup unit 201.

Accordingly, possibility of unnecessary light leaking from a side surface or the like of the light guide cable 202B being made incident on, for example, the light receiving surface of the image pickup unit 201 is conceivable. Therefore, coloring (for example, black) other than transparent is applied to the distal end rigid member 200B in the present embodiment in order to block the leak of the unnecessary light of this type. The other components are the same as the components of the conventional endoscope distal end portion.

As explained above, according to the sixth embodiment, by forming the shape of the distal end portion of the light guide cable 202B configuring the illumination unit as the convex shape 202Ba, it is possible to configure, without providing a lens for illumination, an illumination unit having a function equivalent to a configuration in the case in which the lens for illumination is used, that is, a desired light distribution characteristic. For example, with the configuration explained above, it is possible to obtain light distribution in a wider range without using the lens for illumination.

Therefore, since the number of components can be reduced in the light guide cable 202B functioning as the illumination unit, it is possible to contribute to simplification of an assembly process and, at the same time, contribute to a reduction in manufacturing cost.

Note that the shape of the distal end portion of the light guide cable 202B is not limited to the convex shape 202Ba in the configuration explained above. Desired light distribution can be obtained by changing design of the shape of the distal end portion of the light guide cable 202B as appropriate. For example, the shape of the distal end portion of the light guide cable 202B can be formed as a concave shape 202Bb directed to the rear as shown in FIG. 27. Note that “rear” in this case indicates, for example, a proximal end side (a side closer to the operation section) of the insertion section in the endoscope. With such a configuration as well, a desired light distribution characteristic can be obtained.

In the endoscope distal end portion in the sixth embodiment, as explained above, various internal units, for example, the image pickup unit 201, the illumination unit (the light guide cable 202B), and the treatment instrument channel 203 are disposed inside the distal end rigid member 200B (see FIG. 25 and the like).

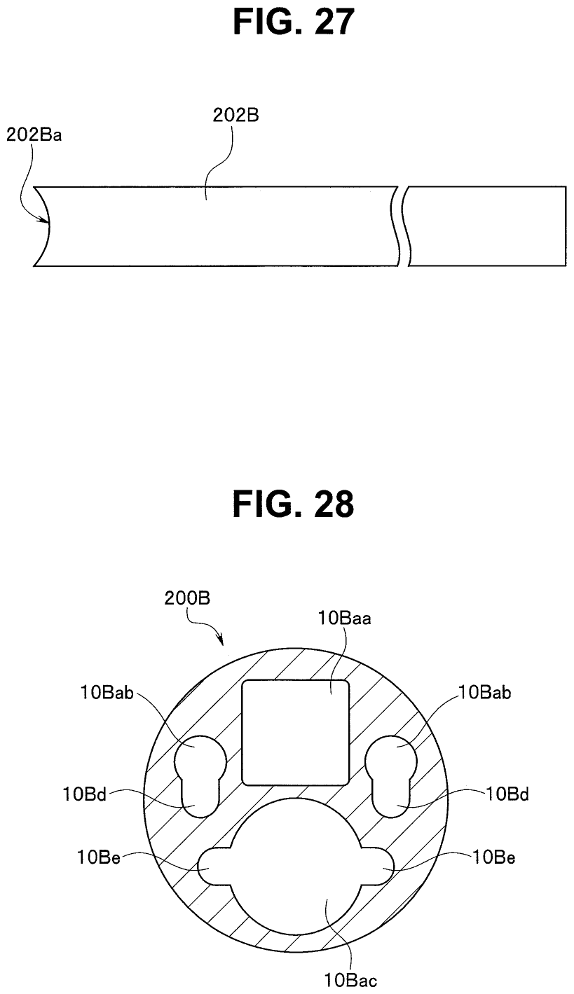

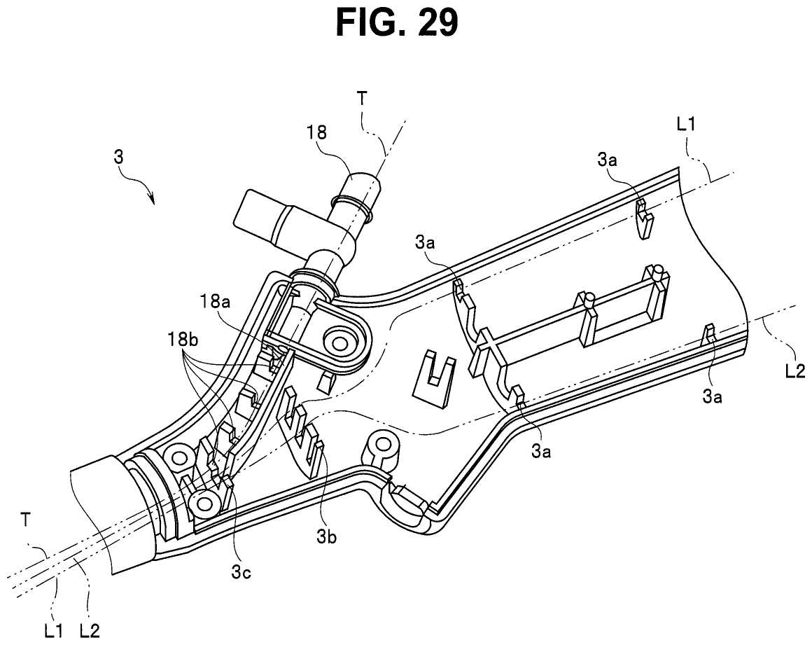

FIG. 28 is a cross-sectional view showing a cut surface indicated by an alternate long and two short dashes line in FIG. 25 viewed in an arrow [28] direction. As shown in FIG. 28, a plurality of disposing sections (10Baa, 10Bab, and 10Bac) having shapes respectively corresponding to the various internal units are formed in the distal end rigid member 200B.

Among these plurality of disposing sections, a part indicated by a sign 10Baa is a disposing section for disposing the image pickup unit 201. Parts indicated by a sign 10Bab are (a plurality of) disposing sections for disposing the illumination units 202. A part indicated by a sign 10Bac is a disposing section for disposing the treatment instrument channel 203.

The plurality of disposing sections (10Baa, 10Bab, and 10Bac) communicate with the observation window 10aa, the illumination window 10ab, and the channel opening 10ac provided on the distal end face of the distal end rigid member 200B and are formed in a through-hole shape toward the rear (closer to the proximal end).

Consequently, an outer surface of the image pickup unit 201 and an inner surface of the disposing section 10Baa are bonded and fixed in a state in which the image pickup unit 201 is disposed in the disposing section 10Baa. Similarly, an outer surface of the light guide cable 202B and an inner surface of the disposing section 10Bab are bonded and fixed in a state in which the distal end portion of the light guide cable 202B is disposed in the disposing section 10Bab. An outer surface of the treatment instrument channel 203 and an inner surface of the disposing section 10Bac are bonded and fixed in a state in which the distal end portion of the treatment instrument channel 203 is disposed in the disposing section 10Bac. Note that an adhesive applied in this case is, for example, an ultraviolet curing adhesive.

Here, the light guide cable 202B and the treatment instrument channel 203 are members formed in an elongated tube shape. Therefore, for example, an inner diameter dimension of the disposing section 10Bab is set to be slightly larger than an outer diameter dimension of the light guide cable 202B. For example, an inner diameter dimension of the disposing section 10Bac is set to be slightly larger than an outer diameter dimension of the treatment instrument channel 203.

When the tubular members (202B and 203) are disposed and bonded in the respective disposing sections (10Bab and 10Bac) corresponding thereto, the adhesive is applied between outer circumferential surfaces of the respective tubular members (202B and 203) and inner surfaces of the respective disposing sections. However, gaps between the tubular members (202B and 203) and inner circumferential surfaces of the respective disposing sections (10Bab and 10Bac) are very small.

Therefore, in the distal end rigid member 200B of the present configuration, as a device for securing an application amount of the adhesive, as shown in FIG. 28 an adhesive storage section 10Bd is formed in a part communicating with the disposing section 10Bab and an adhesive storage section 10Be is formed in a part communicating with the disposing section 10Bac. The adhesive storage sections 10Bd and 10Be are formed in a groove shape communicating from a proximal end side toward a distal end side of the distal end rigid member 200B.

In this way, in the distal end rigid member 200B of the present configuration, since the adhesive storage sections 10Bd and 10Be are provided and formed with respect to the predetermined disposing sections 10Bab and 10Bac, the application amount of the adhesive can be secured more. Therefore, when the various internal units are bonded and fixed inside the distal end rigid member 200B, the bonding can be performed more firmly and in a shorter time.

Various constituent units are disposed inside the operation section in the endoscope. For example, various cables (an image pickup signal cable, a light guide cable, a bending wire, and the like) extended from the insertion section and various tubular members (a suction conduit, an air feeding and water feeding conduit, and the like) are inserted through and disposed inside the operation section in the endoscope. The cables and the tubular members further extend to a universal cable after being inserted through the operation section.

A treatment instrument insertion opening is provided in the operation section. A treatment instrument channel communicates with the treatment instrument insertion opening. Various treatment instruments inserted from the treatment instrument insertion opening are inserted through and disposed in the treatment instrument channel.

In this way, in the endoscope operation section, since a large number of internal components are disposed, the internal components are likely to interfere with one another. However, there is a desire to avoid the interference among the internal components as much as possible.

Therefore, in the endoscope operation section in the present configuration example, a structure explained below is provided as a device for avoiding interference among various constituent units disposed inside.

Here, FIG. 29 is a schematic perspective view showing a part of an internal structure of the endoscope operation section. In FIG. 29, illustration of the various constituent units and the like disposed inside the operation section is omitted and only a part of a housing unit configuring the operation section is shown.

In the endoscope operation section 3 explained in the present configuration example, for example, a plurality of ribs 3a, 3b, and 3c, which are structures for securing disposition paths of the various cables the image pickup signal cable, the light guide cable, and the like; not shown) inserted through an inside of the operation section 3 and preventing interference of the various cables and the other internal components (not shown), are provided. The plurality of ribs 3a, 3b, and 3c are thin plate wall-like sections including one or a plurality of parts where cross sections are formed in a substantially concave shape. Note that the ribs 3a, 3b, and 3c are denoted by different signs and shown because shapes of ribs 3a 3b, and 3c are slightly different as shown in FIG. 29. However, basically, configurations of the ribs only have to include the parts where the cross sections are formed in the substantially concave shape as explained above. The number of substantially concave shape parts and entire shapes of the ribs themselves only have to be set as appropriate according to the cables and the like corresponding to the ribs.

The plurality of ribs 3a, 3b, and 3c are molded integrally with, for example, a housing member of the operation section 3. Without being limited to this configuration, the ribs 3a may be formed of members separate from the operation section 3 and disposed as appropriate in a predetermined position inside the operation section 3 by bonding or the like.

The plurality of ribs 3a are disposed along predetermined disposition paths corresponding to respective predetermined cables. More specifically, for example, there are cables inserted through the insertion section (not shown) and extended to the inside of the operation section 3 and, thereafter, inserted through the operation section 3 and further extended to the universal cable (not shown). Among the cables, for example, a light guide cable and the like are disposed, for example, along alternate long and two short dashes lines added with signs L1 and L2 shown in FIG. 29 inside the operation section 3.

Therefore, the plurality of ribs 3a are disposed side by side at a predetermined interval along a disposition path of the light guide cable. The light guide cable at this time is inserted through among concave portions of the ribs 3a. whereby the disposition path of the light guide cable is secured. At the same time, the light guide cable can avoid interference with the other internal components.

Although illustration and explanation are omitted, about the other cables, the same effects can be obtained if rib members having the same forms as the ribs (3a, 3b, and 3c) are disposed in positions respectively corresponding to the cables and the respective cables are disposed along the rib members.

On the other hand, a treatment instrument (not shown is inserted into the treatment instrument insertion opening 18. The treatment instrument inserted from the treatment instrument insertion opening 18 is configured to be naturally inserted through the treatment instrument channel. Note that, as illustration of a disposition path of the treatment instrument in this case, a path along an alternate long and two short dashes line added with a sign T shown in FIG. 29 is shown.

Therefore, near the treatment instrument insertion opening 18 inside the operation section 3, a guide wall 18a is formed that prevents, when the treatment instrument is inserted into the treatment instrument channel from the treatment instrument insertion opening 18, a distal end of the treatment instrument from moving in directions L1 and L2 in which the light guide cables and the like are disposed in the treatment instrument channel and pressing the light guide cable and the like. Further, a plurality of ribs 18b having substantially the same form as the ribs 3a are disposed side by side art a predetermined interval along the guide wall 18a.

With this configuration, when the treatment instrument inserted from the treatment instrument insertion opening 18 is inserted into the treatment instrument channel, since the treatment instrument is inserted through between the guide wall 18a and the ribs 18b, interference with the other internal components can be avoided.

A technique related to the endoscope 1 in the embodiment explained above is further explained.

### Seventh Embodiment

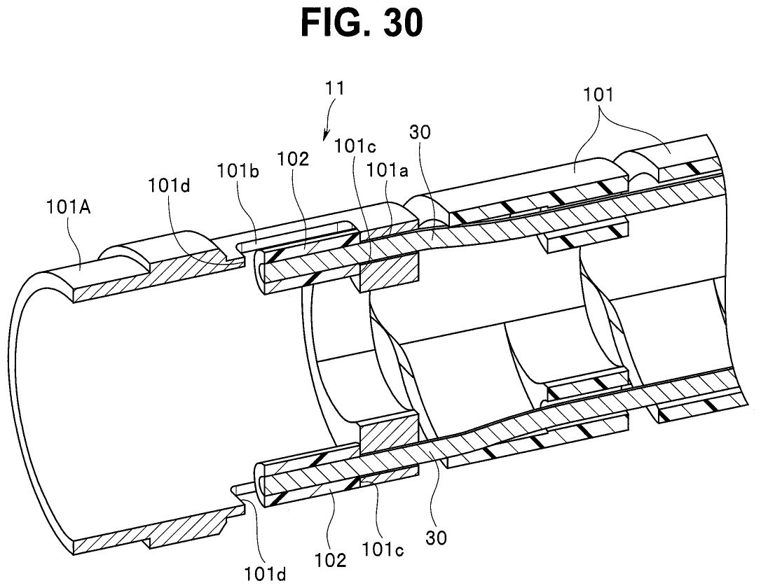

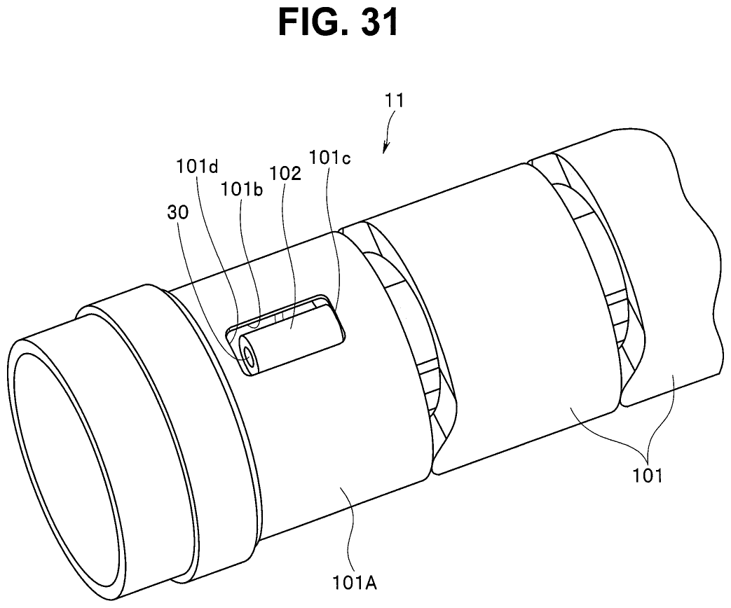

FIG. 30 and FIG. 31 show a seventh embodiment. FIG. 30 is a cross-sectional view showing a configuration example in which a sleeve 102 at a distal end of the wire 30 is connected to a distal end bending piece 101A. FIG. 31 is a perspective view showing a configuration example of a cutout hole 101b provided in the distal end bending piece 101A for inserting the wire 30.

In the seventh embodiment, explanation of the same portions as the portions in the respective embodiments explained above is omitted as appropriate by, for example, adding the same reference numerals and signs to the portions. Only differences are mainly explained.

Conventionally, since a distal end portion of the wire 30 for bending the bending section 11 is fixed to the distal end bending piece 101A by an adhesive, solder, welding, or the like, manhour for assembling the bending section 11 increases and assembly cost increases. Therefore, a configuration example in which a process for fixing the distal end portion of the wire 30 to the distal end bending piece 101A is omitted is explained.

As shown in FIG. 30 and FIG. 31, a plurality of bending pieces 101 are consecutively connected in the bending section 11. The bending piece 101 located at a distalmost end is the distal end bending piece 101A. For example, the cutout hole 101b having a rectangular shape is provided in the distal end bending piece 101A having a tubular shape.

A rear end side (the operation section 3 side of the wire 30 can be inserted through a hole 101a of the distal end bending piece 101A from the cutout hole 101b. The sleeve 102 is integrally provided on a distal end side of the wire 30 by, for example, swaging.

When the sleeve 102 comes into contact with an outlet end face 101c on a distal end side of the hole 101a, the insertion of the wire 30 through the hole 101a ends. At the insertion end time, the entire sleeve 102 is housed in the cutout hole 101b.

The sleeve 102 and the distal end bending piece 101A are not fixed by an adhesive, solder, welding, or the like. Therefore, the wire 30 can slide and move in the hole 101a.

However, movement of the wire 30 to a rear end side in an insertion axis direction is restricted by the contact of the sleeve 102 and the outlet end face 101c of the hole 101a as explained above. Movement of the wire 30 to a distal end side in the insertion axis direction is restricted by the contact of the sleeve 102 and a distal end face 101d of the cutout hole 101b.

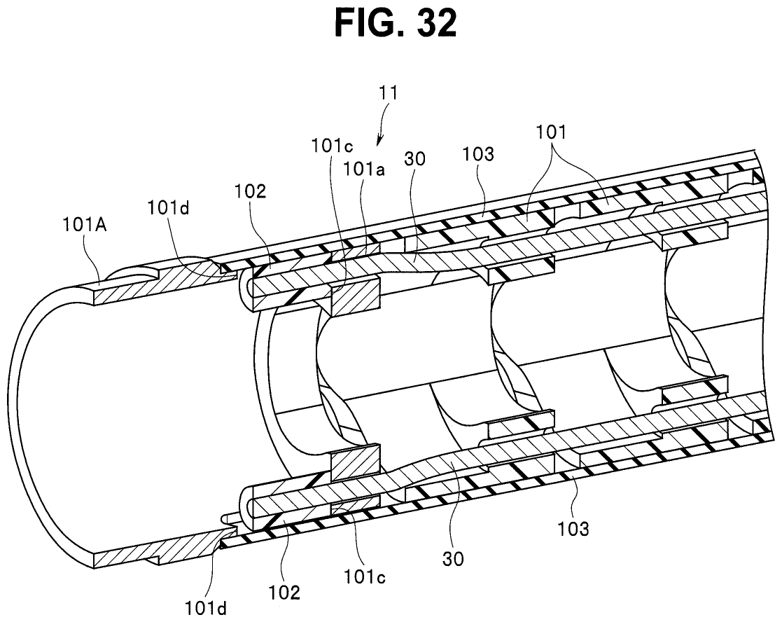

Note that, as shown in FIG. 30 (or FIG. 32 referred to below), a position in a radial direction of the wire 30 in the distal end bending piece 101A is further on an outer diameter side than a position in the radial direction of the wire 30 in the bending piece 101 adjacent to the distal end bending piece 101A. With such a configuration, even if a portion of the distal end face 101d of the distal end bending piece 101A is not increased in thickness, the distal end face 101d can be located on a moving path in the insertion axis direction of the sleeve 102.

According to such a seventh embodiment, since the movement to the distal end side and the rear end side of the wire 30 can be restricted by the sleeve 102, it is possible to prevent positional deviation of the sleeve 102 and prevent angle-down of bending, bending lock, and the like.

Since movement to the distal end side of the wire 30 is restricted, the wire 30 does not extend into the distal end portion 10. The wire 30 can be prevented from coming into contact with the internal components of the distal end portion 10,

Further, since a process for fixing the sleeve 102 to the distal end bending piece 101A with an adhesive, solder, welding, or the like is omitted, the bending section 11 can be inexpensively assembled.

Subsequently, FIG. 32 shows a first modification of the seventh embodiment and is a cross-sectional view showing a configuration example in which the sleeve 102 provided at the distal end of the wire 30 is heat-welded (fused) to a bending section outer skin 103 through the cutout hole 101b.

An outer circumference side of the bending section 11 is covered by the bending section outer skin 103 configured by resin (or rubber or the like).

The bending section outer skin 103 is fixed to the bending section 11 by being heat-welded to a circumferential surface of the distal end bending piece 101A. At this time, the bending section outer skin 103 is heat-welded to the sleeve 102 as well at the same time, the sleeve 102 is fixed integrally with the distal end bending piece 101A. In this case as well, a process for fixing the sleeve 102 and the distal end bending piece 101A with an adhesive, solder, welding, or the like can be omitted.

According to such a first modification of the seventh embodiment, when the bending section outer skin 103 is heat-welded to the circumferential surface of the distal end bending piece 101A, the bending section outer skin 103 is heat-welded to the sleeve 102 as well. Accordingly, it is possible to more surely restrict the sleeve 102 from moving in the insertion axis direction without increasing processes.

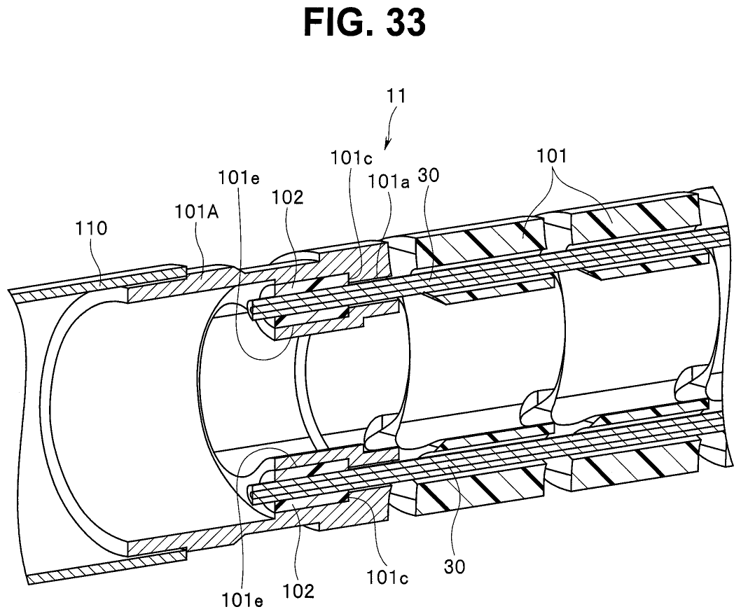

FIG. 33 shows a second modification of the seventh embodiment and is a cross-sectional view showing a configuration example in which the sleeve 102 provided at the distal end of the wire 30 is pressed into an attachment hole 101e provided in the distal end bending piece 101A.

In the distal end bending piece 101A, the attachment hole 101e is provided on a distal end side of the hole 101a through which the wire 30 is inserted. The attachment hole 101e coaxially communicates with the hole 101a. Here, the attachment hole 101e is surrounded by a wall of the distal end bending piece 101A and is larger in a diameter than the hole 101a (however, slightly smaller than an outer diameter of the sleeve 102). Length in the insertion axis direction of the attachment hole 101e is, for example, approximately the same as length in the insertion axis direction of the sleeve 102.

The sleeve 102 is pressed into the attachment hole 101e to fix the sleeve 102 to the distal end bending piece 101A (however, a fixing process by an adhesive, solder, welding, or the like is omitted). Consequently, the sleeve 102 does not move from the attachment hole 101e even if the wire 30 is pulled or slacked.

According to such a second modification of the seventh embodiment, it is possible to more surely restrict the sleeve 102 from moving in the insertion axis direction.

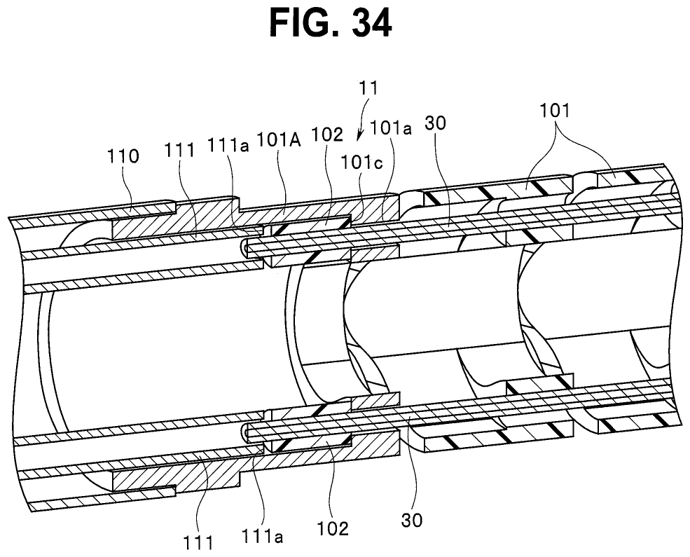

FIG. 34 shows a third modification of the seventh embodiment and is a cross-sectional view showing a configuration example in which the sleeve 102 provided at the distal end of the wire 30 is restricted from moving to a distal end side in the insertion axis direction by a restricting member 111.

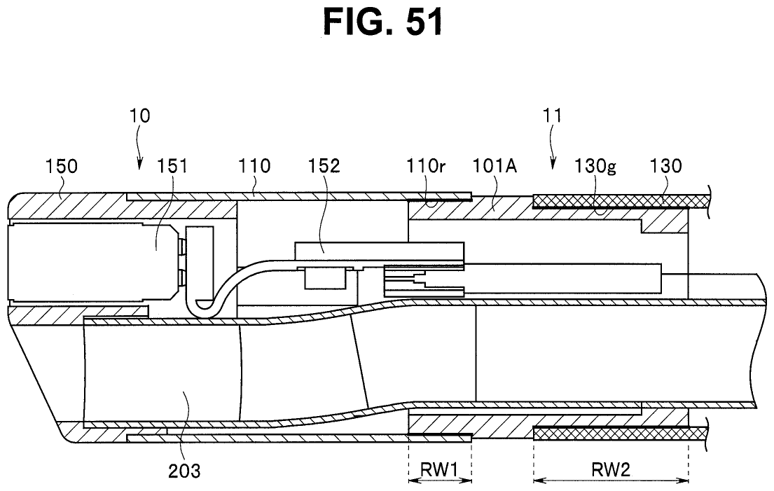

The restricting member 111 formed in, for example, a tubular shape is disposed on a distal end side of the sleeve 102. The restricting member 111 is provided in another member present further on the distal end side than the distal end bending piece 101A and is, for example, configured integrally with a distal end rigid section 150 (see FIG. 51 and the like) in the distal end portion 10.

When a rear end face of the sleeve 102 is in contact with the outlet end face 101c, the restricting member 111 is disposed at a predetermined distance from a distal end face of the sleeve 102 to prevent the distal end face of the sleeve 102 from coming into contact with a rear end face 111a of the restricting member 111.

For example, the distal end portion of the wire 30 projecting from the distal end face of the sleeve 102 is housed in a tubular inside of the restricting member 111.

Note that an example in which the restricting member 111 is formed in the tubular shape is shown in FIG. 34. However, the restricting member 111 is not limited to this and may be formed in, for example, a columnar shape or may be formed in another boss shape or the like.

According to such a third modification of the seventh embodiment, even when the wire 30 is slacked, since the distal end face of the sleeve 102 comes into contact with the rear end face 111a of the restricting member 111, it is possible to prevent positional deviation of the sleeve 102 with respect to the bending section 11. Since the positional deviation of the sleeve 102 is prevented, when the wire 30 is pulled, the rear end face of the sleeve 102 collides with the outlet end face 101c of the distal end bending piece 101A and bending can be surely performed.

### Eighth Embodiment

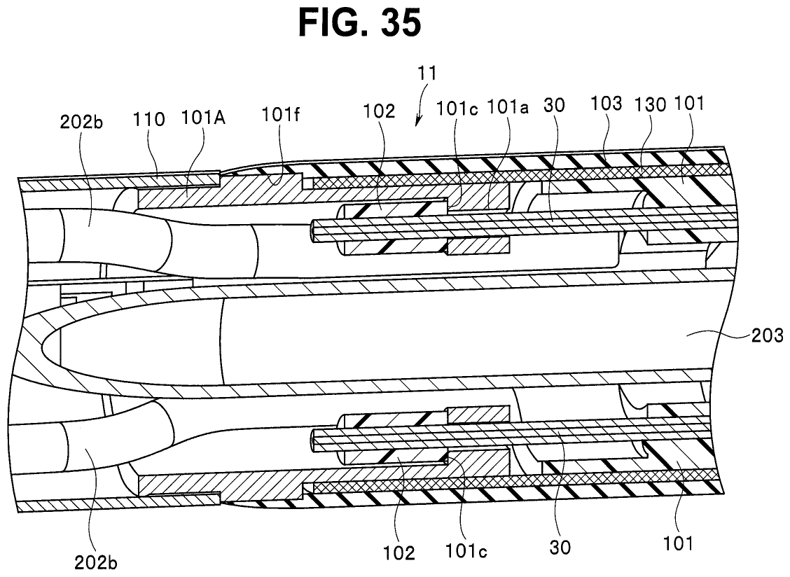

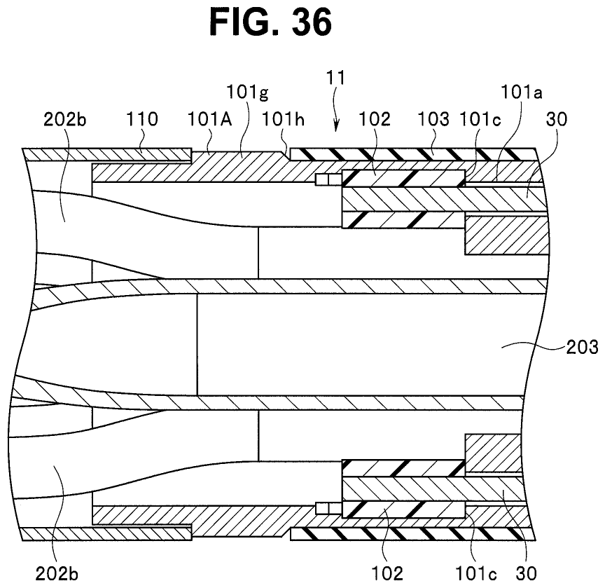

FIG. 35 and FIG. 36 show an eighth embodiment. FIG. 35 is a cross-sectional view showing a configuration example of a rough surface 101f provided on a surface of the distal end bending piece 101A made of metal in order to heat-weld the bending section outer skin 103.

In the eighth embodiment, explanation of the same portions as the portions in the respective embodiments explained above is omitted as appropriate by, for example, adding the same reference numerals and signs to the portions. Only differences are mainly explained.

The wire 30, the light guide cable 202b, the treatment instrument channel 203, and the like are inserted through and, although not shown, an image pickup cable and the like are further inserted through the bending section 11.

Such a distal end bending piece 101A in the bending section 11 needs strength against towing of the wire 30. Therefore, for example, not resin but metal is selected as a material. On the other hand, the bending section outer skin 103 is formed of resin (or rubber or the like). In this case, it is likely that bonding strength of the distal end bending piece 101A and the bending section outer skin 103 decreases and the bending section outer skin 103 is turned up.

Therefore, as shown in FIG. 35, roughening (treatment for roughening a surface) is performed on an outer circumferential surface, which is in contact with the bending section outer skin 103, of the distal end bending piece 101A formed of metal by chemical treatment, mechanical treatment, or the like to form the rough surface 101f. The bending section outer skin 103 is heat-welded to the rough surface 101f of the distal end bending piece 101A. Note that a bending section braid 130 is disposed on an inner circumference side of the bending section outer skin 103 further on a rear end side than the rough surface 101f.

FIG. 36 is a cross-sectional view showing a configuration example in which a cut surface 101h is provided on a rear end side of a circumferential convex portion 101g of the distal end bending piece 101A. As illustrated, the cut surface 101h is configured as, for example, a chamfered slope corresponding to a height of the circumferential convex portion 101g and is a structure for making heat-welding of the bending section outer skin 103 easy and reliable.

According to such an eighth embodiment, since the rough surface 101f is provided in the distal end bending piece 101A and the bending section outer skin 103 is heat-welded, connection strength of the bending section outer skin 103 to the distal end bending piece 101A can be increased. In this way, even if a process for, for example, winding and fixing a thread on a distal end side of the bending section outer skin 103 is omitted, it is possible to prevent the bending section outer skin 103 from being turned up.

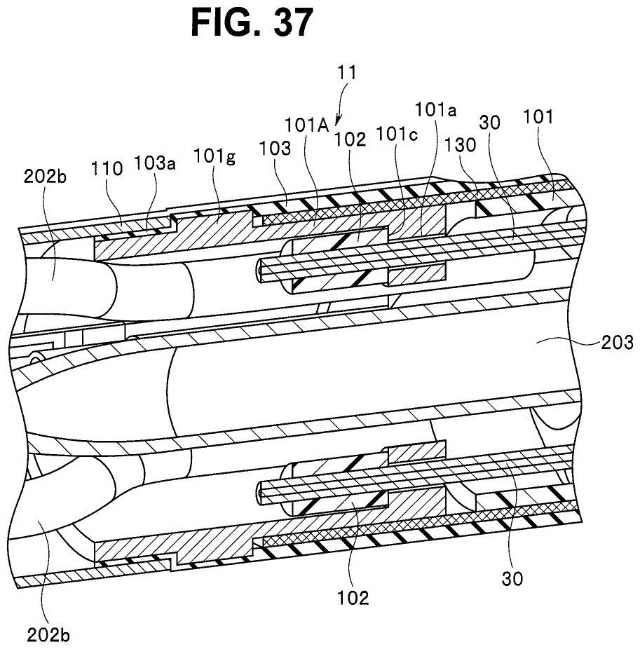

FIG. 37 shows a modification of the eighth embodiment and is a cross-sectional view showing a configuration example in which the bending section outer skin 103 is sandwiched by the connection tube 110 and the distal end bending piece 101A.

In the configuration example shown in FIG. 37, further on the distal end side than the circumferential convex portion 101g, the distal end portion 103a of the bending section outer skin 103 is sandwiched between the distal end bending piece 101A on an inner circumference side and the connection tube 110 on an outer circumference side. Here, the connection tube 110 is a tubular member formed of metal or the like that connects the distal end bending piece 101A and the distal end rigid section 150 in the distal end portion 10 (see FIG. 51 and the like).

According to such a modification of the eighth embodiment, by sandwiching the distal end portion 103a of the bending section outer skin 103 between the distal end bending piece 101A and the connection tube 110 as well, it is possible to surely prevent the bending section outer skin 103 from being turned up.

### Ninth Embodiment

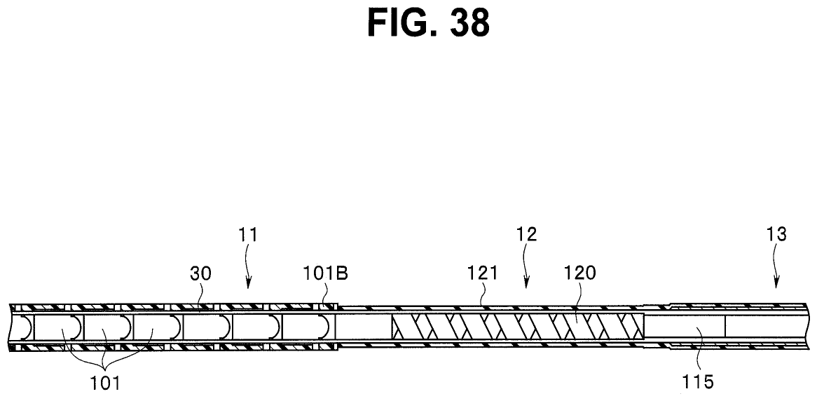

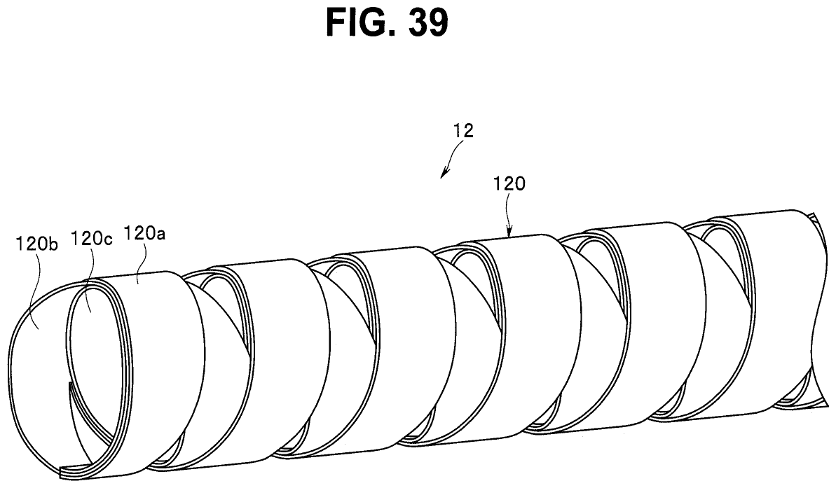

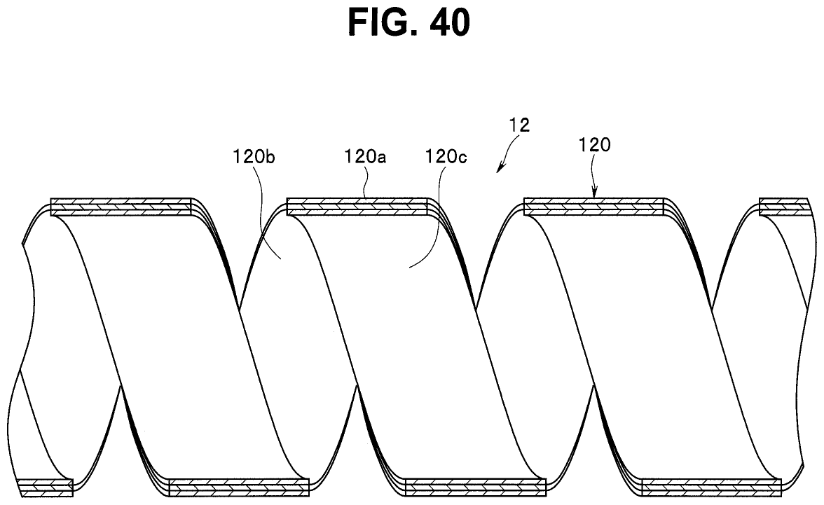

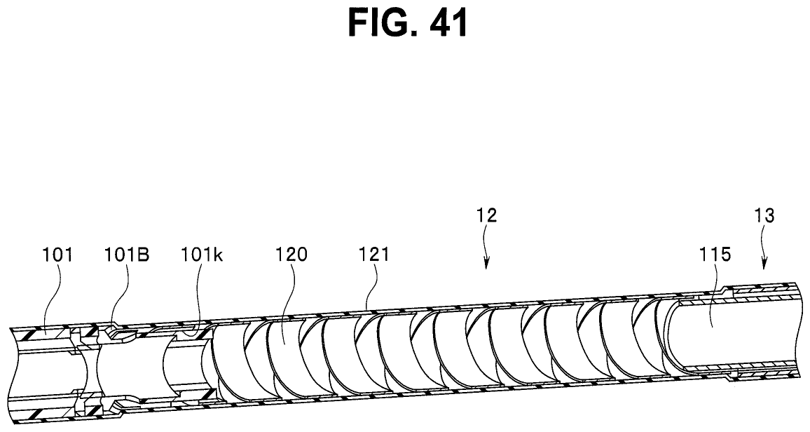

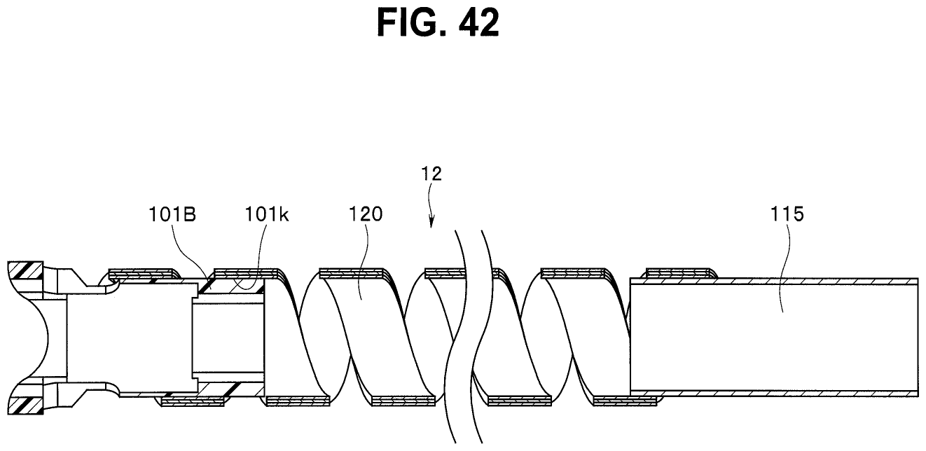

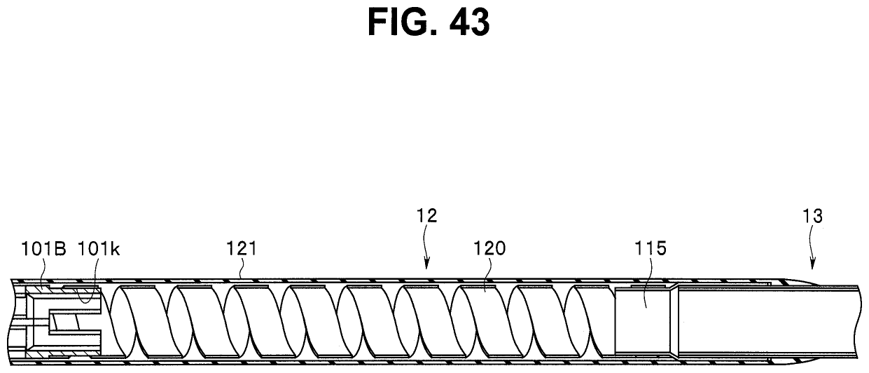

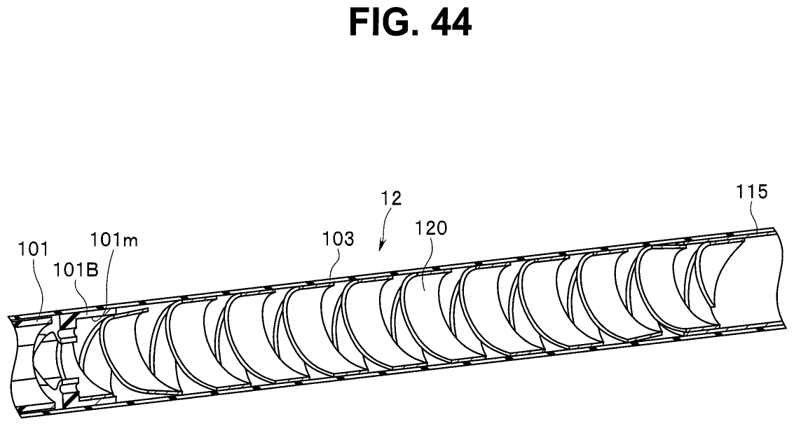

FIG. 38 to FIG. 44 show a ninth embodiment. FIG. 38 is a cross-sectional view showing a configuration example of the passive bending section 12 provided between the bending section 11 and the flexible tube section 13. FIG. 39 is a perspective view showing a configuration example of a three-layer flexible tube 120. FIG. 40 is a cross-sectional view showing the configuration example of the three-layer flexible tube 120. FIG. 41 is a cross-sectional view showing an example in which the passive bending section 12 is configured by covering the three-layer flexible tube 120 with a passive bending section outer skin 121. FIG. 42 is a cross-sectional view showing a configuration example in which a distal end portion of the three-layer flexible tube 120 is connected to an outer circumferential surface 101k of a rear end bending piece 101B and a rear end portion of the three-layer flexible tube 120 is connected to an outer circumferential surface of a front pipe sleeve 115 of the flexible tube section 13. FIG. 43 is a cross-sectional view showing a configuration example in which the connection of the distal end portion of the three-layer flexible tube 120 and the outer circumferential surface 101k of the rear end bending piece 101B and the connection of the rear end portion of the three-layer flexible tube 120 and an outer circumferential surface of the front pipe sleeve 115 of the flexible tube section 13 are performed by laser welding. FIG. 44 is a cross-sectional view showing a configuration example in which the distal end portion of the three-layer flexible tube 120 is connected to an inner circumferential surface 101m of the rear end bending piece 101B and the rear end portion of the three-layer flexible tube 120 is connected to an inner circumferential surface of the front pipe sleeve 115 of the flexible tube section 13.

In the ninth embodiment, explanation of the same portions as the portions in the respective embodiments explained above is omitted as appropriate by, for example, adding the same reference numerals and signs to the portions. Only differences are mainly explained.

As shown in FIG. 1 and FIG. 38, the passive bending section 12 is provided between the bending section 11 and the flexible tube section 13. The passive bending section 12 is a part that passively bends when receiving force from an outside.

It is preferable that it should be possible to inexpensively and easily assemble such a passive bending section 12 while securing torque followability. The passive bending section 12 is also requested to be small in an outer diameter. Further, the passive bending section 12 is desirably configured to be able to reduce an amount of force at the time when bending operation for the bending section 11 is performed by the bending operation lever 15 (or not increase the amount of force).

Therefore, as shown in FIG. 39 and FIG. 40, the passive bending section 12 in the present embodiment is configured not by a conventional one-layer flexible tube but by the three-layer flexible tube 120.

In other words, the three-layer flexible tube 120 is formed of, for example, metal and formed in a three-layer structure including an outer layer flexible tube 120a, a middle layer flexible tube 120b, and an inner layer flexible tube 120c. The outer layer flexible tube 120a, the middle layer flexible tube 120b, and the inner layer flexible tube 120c are configured in order of right-handed winding, left-handed winding, and right-handed winding or configured in order of left-handed winding, right-handed winding, and left-handed winding.

The outer layer flexible tube 120a, the middle layer flexible tube 120b, and the inner layer flexible tube 120c may be respectively differentiated in widths and thicknesses of spirally wound materials and pitches in winding the materials. By appropriately selecting the widths, the thicknesses, and the pitches of the respective layers, it is possible to adjust, as desired, bending stress necessary for the passive bending section 12. Therefore, by using the adjusted three-layer flexible tube 120, it is possible to configure the passive bending section 12 having preferable characteristics corresponding to an applied field of the endoscope 1.

Further, as shown in FIG. 41, the passive bending section outer skin 121 configured by resin (for example, PETF) is provided by, for example, integral molding on an outer circumference side of the three-layer flexible tube 120. However, a conventional net-like braid for imparting torque followability is not provided on the outer circumference side of the three-layer flexible tube 120. Therefore, the passive bending section 12 is formed in a braid-less structure.

The three-layer flexible tube 120 is molded to be longer in the insertion axis direction than the passive bending section outer skin 121. Consequently, both end portions of the three-layer flexible tube 120 extend from both ends of the passive bending section outer skin 121 to enable laser welding.

In an example shown in FIG. 41 to FIG. 43, the distal end portion (including a cut end portion) of the three-layer flexible tube 120 is connected to the outer circumferential surface 101k of the rear end bending piece 101B formed of metal. Here, the rear end bending piece 101B is the bending piece 101 located on a rearmost side among the plurality of bending pieces 101 consecutively connected in the bending section 11.

The rear end portion (including a cut end portion) of the three-layer flexible tube 120 is connected to the outer circumferential surface of the front pipe sleeve 115 (made of metal) provided on a distal end side of the flexible tube section 13.

In order to make these connections reliable, an inner diameter of the three-layer flexible tube 120 alone (an inner diameter of the inner layer flexible tube 120c) is set to be smaller than an outer diameter of the outer circumferential surface 101k of the rear end bending piece 101B and smaller than an outer diameter of the front pipe sleeve 115.

The distal end portion of the three-layer flexible tube 120 is expanded in diameter and then assembled to the rear end bending piece 101B and, thereafter, a portion including the cut end portion is connected to the rear end bending piece 101B by, for example, laser welding.

Similarly, the rear end portion of the three-layer flexible tube 120 is expanded in diameter and then assembled to the front pipe sleeve 115 and, thereafter, a portion including the cut end portion is connected to the front pipe sleeve 115 by, for example, laser welding.

Consequently, the three-layer flexible tube 120 is integrated with the rear end bending piece 101B at the distal end portion and integrated with the front pipe sleeve 115 at the rear end portion.

On the other hand, in an example shown in FIG. 44, the distal end portion including the cut end portion) of the three-layer flexible tube 120 is connected to the inner circumferential surface 101m of the rear end bending piece 101B. The rear end portion (including the cut end portion) of the three-layer flexible tube 120 is connected to the inner circumferential surface of the front pipe sleeve 115 provided on the distal end side of the flexible tube section 13,

In order to make these connections reliable, an outer diameter of the three-layer flexible tube 120 alone (an outer diameter of the outer layer flexible tube 120a) is set to be lamer than an inner diameter of the inner circumferential surface 101m of the rear end bending piece 101B and larger than an inner diameter of the front pipe sleeve 115.

The distal end portion of the three-layer flexible tube 120 is contracted in diameter and then assembled to the rear end bending piece 101B and, thereafter, a portion including the cut end portion is connected to the rear end bending piece 101B by, for example, laser welding.

Similarly, the rear end portion of the three-layer flexible tube 120 is contracted in diameter and then assembled to the front pipe sleeve 115 and, thereafter, a portion including the cut end portion is connected to the front pipe sleeve 115 by, for example, laser welding.

Consequently, the three-layer flexible tube 120 is integrated with the rear end bending piece 101B at the distal end portion and integrated with the front pipe sleeve 115 at the rear end portion.

According to such a ninth embodiment, since the passive bending section 12 is configured by the three-layer flexible tube 120, it is possible to omit the net-like braid in the passive bending section outer skin 121 while securing torque followability. Since the net-like braid is omitted, it is possible to reduce a price and facilitate assembly. Further, with a braid-less configuration, it is possible to reduce an outer diameter of the passive bending section 12 and reduce an amount of force of bending operation of the bending section 11 by the bending operation lever 15.

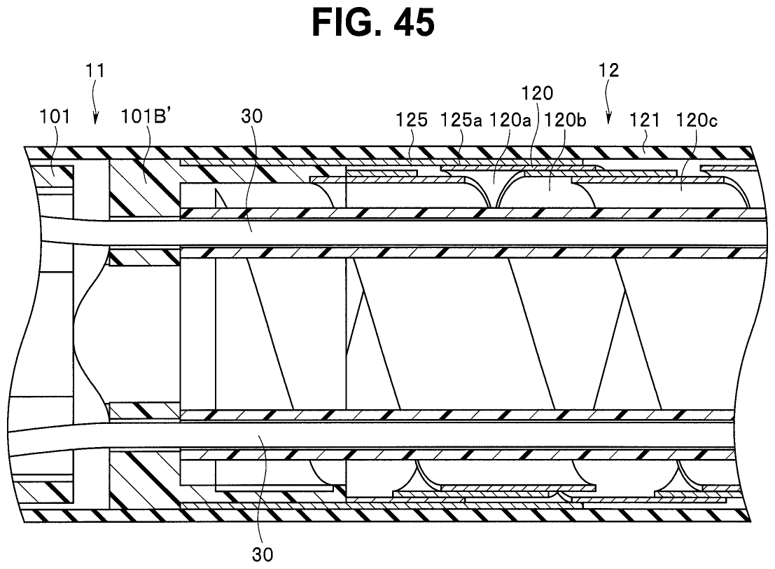

FIG. 45 shows a first modification of the ninth embodiment and is a cross-sectional view showing a configuration example in which a rear end bending piece 101B′ is insert-molded in a metal pipe 125 and the three-layer flexible tube 120 of the passive bending section 12 is laser-welded to the metal pipe 125.

In order to laser-weld the three-layer flexible tube 120, as explained above, there is a method of forming the rear end bending piece 101B with metal. However, since the rear end bending piece 101B is complicated in shape, cost is required if the rear end bending piece 101B is formed of metal.

Therefore, as shown in FIG. 45, the rear end bending piece 101B′ is inserted-molded in the metal pipe 125 with resin. Consequently, a complicated shape portion can be configured by the resin. Only the metal pipe 125 having a simple shape has to be formed of metal.

The distal end portion including the cut end portion of the three-layer flexible tube 120 is laser-welded to, for example, an inner circumferential surface 125a (or an external circumferential surface) of the metal pipe 125, whereby the three-layer flexible tube 120 is integrated with the metal pipe 125 and is integrated with the rear end bending piece 101B′.

According to such a first modification of the ninth embodiment, it is possible to simplify assembly, reduce cost, and prevent the cut end portion of the three-layer flexible tube 120 from separating from the metal pipe 125.

Note that the rear end bending piece 101B′ may be insert-molded in the three-layer flexible tube 120 itself with resin without using the metal pipe 125. In this case, it is possible to further reduce cost.

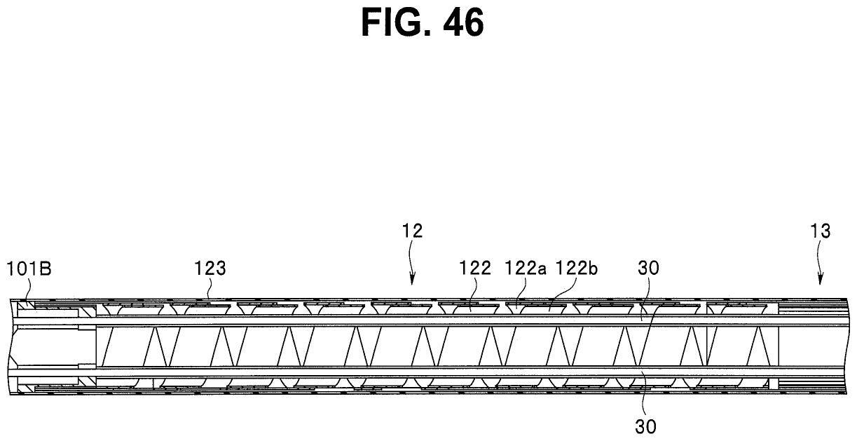

FIG. 46 shows a second modification of the ninth embodiment and is a cross-sectional view showing a configuration example in which a two-layer flexible tube 122 and a heat shrinkable tube 123 are used in the passive bending section 12.

As another example of a configuration in which the outer diameter of the passive bending section 12 is reduced, as shown in FIG. 46, the two-layer flexible tube 122 and the heat shrinkable tube 123 may be used.

The two-layer flexible tube 122 is formed of, for example, metal and is formed in a two-layer structure of an outer layer flexible tube 122a and an inner layer flexible tube 122b. Here, the outer layer flexible tube 122a and the inner layer flexible tube 122b are configured in order of right-handed winding and left-handed winding or configured in order of left-handed winding and right-handed winding.

The heat shrinkable tube 123 for securing torque followability is disposed on an outer circumference side of the two-layer flexible tube 122. The heat shrinkable tube 123 is integrated with the two-layer flexible tube 122 by applying heat thereto.

According to such a second modification of the ninth embodiment as well, it is possible to secure torque followability while achieving a braid-less structure and achieve a reduction in a diameter of the passive bending section 12.

### Tenth Embodiment

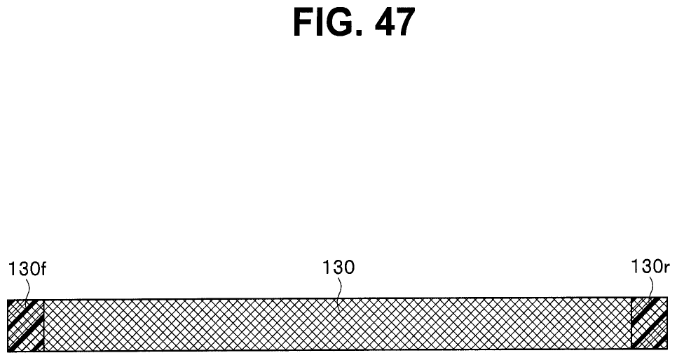

FIG. 47 shows a tenth embodiment and is a side view shooing a configuration example of the bending section braid 130.

In the tenth embodiment, explanation of the same portions as the portions in the respective embodiments explained above is omitted as appropriate by, for example, adding the same reference numerals and signs to the portions. Only differences are mainly explained.

The bending section braid 130 is disposed on an outer circumference side of the bending piece 101 (including the distal end bending piece 101A and the rear end bending piece 101B or 101B′) of the bending section 11 in order to increase torsional strength of the bending section 11. Since the bending section braid 130 is configured as a net made of metal, a process such as soldering is necessary to fix the bending section braid 130 to the bending section 11 and assembly cost increases.

A configuration shown in FIG. 47 is devised considering such a point.

In other words, resin is contained in a distal end portion 130f and a rear end portion 130r of the bending section braid 130.

A resin portion of the distal end portion 130f of the bending section braid 130 having such a configuration can be fixed to a member on a distal end side by being heat-welded and a resin portion of the rear end portion 130r of the bending section braid 130 can be fixed to a member on a rear end side by being heat-welded.

Note that the distal end portion 130f is preferably present only further on the distal end side than between the distal end bending piece 101A and the bending piece 101 adjacent to the distal end bending piece 101A. Similarly, the rear end portion 130r is preferably present only further on the rear end side than between the rear end bending piece 101B or 101B′ and the bending piece 101 adjacent to the rear end bending piece 101B or 101B′. If such a configuration is adopted, it is possible to prevent, with resin, an amount of force of bending operation from increasing.

According to such a tenth embodiment, since the resin is contained in the distal end portion 130f and the rear end portion 130r of the bending section braid 130, it is possible to fix the bending section braid 130 if the resin is heat-welded. Consequently, the soldering processing is unnecessary and the fixing work is facilitated. Assembly cost can be reduced.

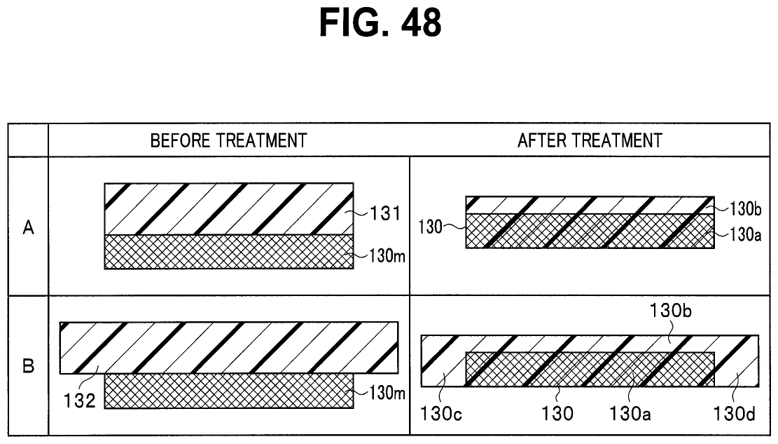

FIG. 48 shows a modification of the tenth embodiment and is a diagram showing a processing example for manufacturing the bending section braid 130. Note that cross sections of main parts are shown in respective fields of FIG. 48.

An A field of FIG. 48 shows a first method of manufacturing the bending section braid 130. The bending section braid 130 in which a braid material 130m and a resin material 131 are integrated is formed by superimposing, on the braid material 130m, the resin material 131 having the same length in the insertion axis direction as the length of the braid material 130m and applying heat and laminating the resin material 131 and the braid material 130m.

In the bending section braid 130 formed in this way, a resin layer 130b is formed on a braid layer 130a containing resin.

An B field of FIG. 48 shows a second method of manufacturing the bending section braid 130. The bending section braid 130 in which the braid material 130m and the resin material 131 are integrated is formed by superimposing, on the braid material 130m, a resin material 132 having length in the insertion axis direction larger than the length of the braid material 130m such that both ends of the resin material 132 protrude and applying heat and laminating the resin material 131 and the braid material 130m.

In the bending section braid 130 formed in this way, the resin layer 130b is formed on the braid layer 130a containing resin and a resin section 130c not including the braid material 130m on a distal end side and a resin section 130d not including the braid material 130m on a rear end side are formed.

According to such a modification of the tenth embodiment as well, it is possible to fix the bending section braid 130 with heat welding, fixing work is facilitated, and assembly cost can be reduced. Since the resin layer 130b is formed integrally with the bending section braid 130, it is unnecessary to provide a passive bending section outer skin as another component and a manufacturing process can be simplified.

Further, since the resin is laminated, it is possible to secure torque followability of the bending section 11 and prevent the bending section braid 130 from protruding in an outer diameter direction.

### Eleventh Embodiment

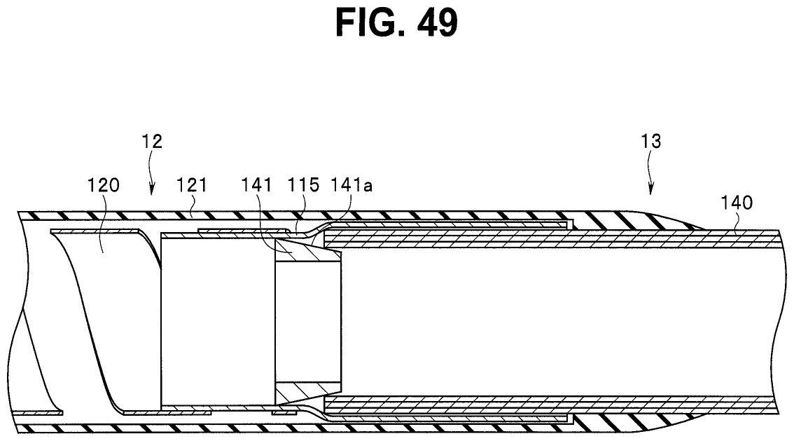

FIG. 49 shows an eleventh embodiment and is a cross-sectional view showing a configuration example in which a flexible tube section braid 140 is internally inserted into the front pipe sleeve 115 of the flexible tube section 13 and the flexible tube section braid 140 is locked in an outer diameter direction by a taper member 141.

In the eleventh embodiment, explanation of the same portions as the portions in the respective embodiments explained above is omitted as appropriate by, for example, adding the same reference numerals and signs to the portions. Only differences are mainly explained.

The flexible tube section braid 140 is formed in, for example, a three-layer structure in which both surfaces of a braid are sandwiched by resin.

When such a flexible tube section braid 140 is heat-welded to an inner diameter side of the front pipe sleeve 115, the flexible tube section braid 140 sometimes protrudes to the inner diameter side in a portion where a diameter of the front pipe sleeve 115 changes.

Therefore, as shown in FIG. 49, the taper member 141 provided with a taper surface 141a on an outer circumference side is provided and the flexible tube section braid 140 is locked in the outer diameter direction by the taper surface 141a.

When the flexible tube section braid 140 is heat-welded to an inner diameter side of the front pipe sleeve 115, the taper member 141 is simultaneously heat-welded.

According to such an eleventh embodiment, it is possible to prevent the flexible tube section braid 140 from protruding to the inner diameter side. Therefore, the flexible tube section braid 140 does not come into contact with internal components. Damage to the internal components can be prevented.

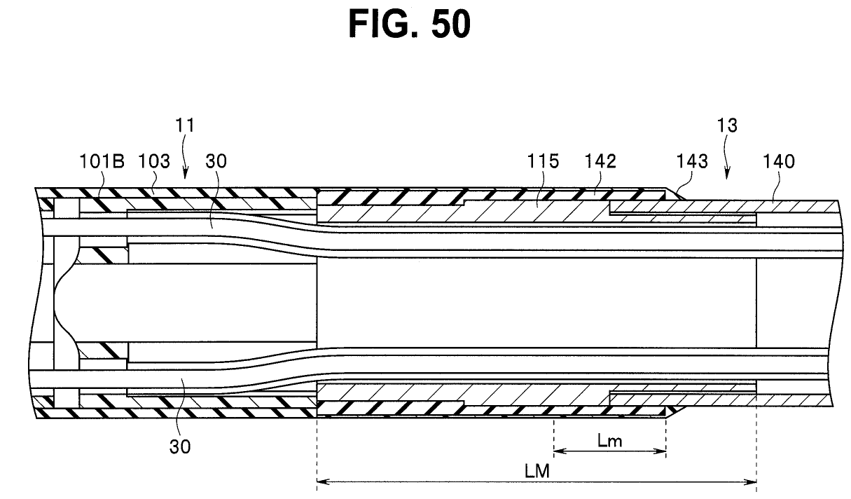

FIG. 50 shows a modification of the eleventh embodiment and is a cross-sectional view showing a configuration example in which the flexible tube section braid 140 is externally inserted onto the front pipe sleeve 115 of the flexible tube section 13 and the flexible tube section braid 140 is locked in an inner diameter direction by a resin tube.

When the flexible tube section braid 140 is connected to an outer diameter side of the front pipe sleeve 115, an end portion of the flexible tube section braid 140 sometimes protrudes to the outer diameter side.

Therefore, the end portion of the flexible tube section braid 140 is covered by a resin tube 142 and the resin tube 142 is heat-welded or heat-shrunk to thereby hold the end portion of the flexible tube section braid 140 not to protrude to the outer diameter side.

Here, it is sufficient that length in an insertion axis direction of the resin tube 142 is set to be equal to or larger than a length Lm that can cover at least a distal end of the flexible tube section braid 140 and a part of the front pipe sleeve 115 and equal to or smaller than a length LM that can cover the entire front pipe sleeve 115.

Further, in a configuration shown in FIG. 50, a resin layer 143 is provided on an outer circumference side of the resin tube 142 to make a diameter change at an end portion of the resin tube 142 gentle.

Note that, in FIG. 50, the configuration example is shown in which the bending section 11 is consecutively connected to the distal end side of the flexible tube section 13 and the passive bending section 12 is omitted. However, the configuration can also be applied to a configuration in which the passive bending section 12 is provided.

Naturally, the same configuration may be applied to the bending section braid 130 disposed on the outer circumference side of the bending section 11.

According to such a modification of the eleventh embodiment, it is possible to prevent the flexible tube section braid 140 (or the bending section braid 130) from protruding to the outer diameter side.

### Twelfth Embodiment

FIG. 51 shows a twelfth embodiment and is a sectional view showing a configuration example in which the connection tube 110 and the bending section braid 130 are laser-welded to the distal end bending piece 101A.

In the twelfth embodiment, explanation of the same portions as the portions in the respective embodiments explained above is omitted as appropriate by, for example, adding the same reference numerals and signs to the portions. Only differences are mainly explained.

The distal end portion 10 and the bending section 11 of the endoscope 1 are desirably assembled as inexpensively as possible. A configuration example for such an assembly is explained.

Internal components such as the distal end rigid section 150, an image pickup unit 151 including an image pickup lens and an image pickup device, a circuit board 152 electrically connected to the image pickup unit 151, and the treatment instrument channel 203 are disposed at the distal end portion 10 of the endoscope 1.

The connection tube 110 made of metal is disposed between a rear end side of an outer circumference of the distal end rigid section 150 and a distal end side of an outer circumference of the distal end bending piece 101A made of metal. A rear end portion 110r of the connection tube 110 made of metal is connected to the distal end bending piece 101A made of metal by laser welding in an insertion axis direction range RW1.

When the bending section braid 130 made of metal is provided in the bending section 11, a distal end portion 130g of the bending section braid 130 is connected to the distal end bending piece 101A made of metal by laser welding in an insertion axis direction range RW2. At this time, a manufacturing process can be simplified by collectively performing the laser welding of the connection tube 110 and the laser welding of the bending section braid 130 in the same process.

According to such a twelfth embodiment, since the connection tube 110 and the bending section braid 130 are collectively laser-welded to the distal end bending piece 101A, it is possible to simplify the manufacturing process and reduce assembly cost.

Further, the present invention is not limited to the embodiments explained above and can be changed as appropriate in a range not contrary to the gist or the idea of an invention that is read from the claims, the entire specification, and the drawings.

What is claimed is:

1. A bending section of an endoscope bent by a wire being towed, the bending section being provided in an endoscope insertion section, the bending section comprising at least one first bending piece and at least one second bending piece, each of the first bending piece and the second bending piece being tubular and including a hole formed along a longitudinal axis of the endoscope insertion section, an internal component of the endoscope being disposed in the hole, wherein
the first bending piece includes a first groove communicating with the hole, formed from an inner circumference of the first bending piece toward an outer side of a radial direction of the longitudinal axis, and formed at a width substantially equal to an outer diameter of the wire and a depth equal to or larger than the outer diameter of the wire,
the second bending piece is disposed adjacent to the first bending piece along the longitudinal axis, and the second bending piece includes a second groove formed from an outer circumference of the second bending piece toward the hole and formed at a width substantially equal to the outer diameter of the wire and a. depth equal to or larger than the outer diameter of the wire, and
the wire is disposed in the first groove and the second groove.

2. The bending section of the endoscope according to claim 1, wherein
the first groove includes, along the longitudinal axis, a distal end portion, a proximal end portion, and an intermediate portion provided between the distal end portion and the proximal end portion, and
the distal end portion or the proximal end portion is formed deeper than the intermediate portion.

3. The bending section of the endoscope according to claim 1, wherein
the second groove includes, along the longitudinal axis, a distal end portion, a proximal end portion, and an intermediate portion provided between the distal end portion and the proximal end portion, and
the distal end portion or the proximal end portion is formed deeper than the intermediate portion.

4. The bending section of the endoscope according to claim 1, wherein the first groove and the second groove are formed such that a position in the radial direction of the wire is disposed further on the outer side of the radial direction in the second groove than in the first groove.

5. The bending section of the endoscope according to claim 4, wherein
the first groove and the second groove each include, along the longitudinal axis, a distal end portion, a proximal end portion, and an intermediate portion provided between the distal end portion and the proximal end portion, and
a position in the radial direction of the wire is disposed further on the outer side of the radial direction in the intermediate portion of the second groove than in the intermediate portion of the first groove.

6. An endoscope insertion section formed in a tube shape, the endoscope insertion section comprising a bending section bent by a wire being towed and including at least one first bending piece and at least one second bending piece, each of the first bending piece and the second bending piece being tubular and including a hole formed along a longitudinal axis of the endoscope insertion section, an internal component of the endoscope being disposed in the hole, wherein
the first bending piece includes a first groove communicating with the hole, formed from an inner circumference of the first bending piece toward an outer side of a radial direction of the longitudinal axis, and formed at a width substantially equal to an outer diameter of the wire and a depth equal to or larger than the outer diameter of the wire,
the second bending piece is disposed adjacent to the first bending piece along the longitudinal axis, and the second bending piece includes a second groove formed from an outer circumference of the second bending piece toward the hole and formed at a width substantially equal to the outer diameter of the wire and a depth equal to or larger than the outer diameter of the wire, and
the wire is disposed in the first groove and the second groove.

7. An endoscope comprising an endoscope insertion section formed in a tube shape, the endoscope insertion section including a bending section bent by a wire being towed and including at least one first bending piece and at least one second bending piece, each of the first bending piece and the second bending piece being tubular and including a hole formed along a longitudinal axis of the endoscope insertion section, an internal component of the endoscope being disposed in the hole, wherein
the first bending piece includes a first groove communicating with the hole, formed from an inner circumference of the first bending piece toward an outer side of a radial direction of the longitudinal axis, and formed at a width substantially equal to an outer diameter of the wire and a depth equal to or larger than the outer diameter of the wire,
the second bending piece is disposed adjacent to the first bending piece along the longitudinal axis, and the second bending piece includes a second groove formed from an outer circumference of the second bending piece toward the hole and formed at a width substantially equal to the outer diameter of the wire and a depth equal to or larger than the outer diameter of the wire, and
the wire is disposed in the first groove and the second groove.
